(19) United States
(12) Patent Application Publication
(10) Pub. No.: US 2025/0221984 A1
(43) Pub. Date: Jul. 10, 2025

(54) TOPICAL ADMINISTRATION TO THE ORAL CAVITY

(71) Applicant: CMPD Licensing, LLC, Conroe, TX (US)

(72) Inventor: Jay Richard Ray, II, Conroe, TX (US)

(73) Assignee: CMPD Licensing, LLC, Conroe, TX (US)

(21) Appl. No.: 19/067,153

(22) Filed: Feb. 28, 2025

Related U.S. Application Data
(63) Continuation-in-part of application No. 18/236,300, filed on Aug. 21, 2023.

(51) Int. Cl. A61K 31/485; A61K 9/00; A61K 9/107; A61K 9/127; A61K 31/4045; A61K 31/78; A61K 47/14; A61K 47/24; A61K 47/38

(52) U.S. Cl. CPC A61K 31/485; A61K 9/006; A61K 9/1075; A61K 9/127; A61K 31/4045; A61K 31/78; A61K 47/14; A61K 47/24; A61K 47/38

(57) ABSTRACT

A method of treating opioid use disorder (OUD) or alcohol use disorder (AUD) includes administering a pharmaceutically effective amount of a topical composition comprising an anhydrous suspension including naltrexone to an oral cavity of a subject for oral absorption therein. The topical composition is formulated to self-emulsify in an aqueous environment of the oral cavity.

### CROSS-REFERENCE TO RELATED APPLICATIONS

present application is a continuation-in-part of U.S. patent application Ser. No. 18/236,300, filed Aug. 21, 2023, the content of which is hereby incorporated herein by reference.

### TECHNOLOGY

The present disclosure relates to compositions and methods of making and using compositions for topical administration generally by transmucosal delivery.

### SUMMARY

In one aspect, a topical composition for administration to an oral cavity of a subject and oral absorption therein to treat opioid use disorder (OUD) or alcohol use disorder (AUD) includes an anhydrous suspension. The anhydrous liquid suspension may include naltrexone, medium chain triglycerides, between 0.5% w/w and 10% w/w phosphatidylcholine, between 0.1% w/w and 1% w/w lysophosphatidylcholine, glyceryl distearate, and glyceryl monostearate. The anhydrous suspension is configured to self-emulsify in an aqueous environment of the oral cavity of the subject to form a mixed micelle and liposome delivery system for transmucosal delivery of the naltrexone within the oral cavity.

In one example, the medium chain triglycerides comprise caprylic/capric triglycerides. The caprylic/capric triglycerides may be present in an amount between 60% and 95% w/w. The caprylic/capric triglycerides may be present in an amount between 50% and 81% w/w.

In one example, the anhydrous suspension further comprises polycarbophil in an amount between 0.2% and 2% w/w.

In one example, the anhydrous suspension further comprises polycarbophil in an amount between 0.17% and 2% w/w.

In one example, the anhydrous suspension further comprising peppermint flavor, spearmint flavor, or both.

In one example, the glyceryl distearate and glyceryl monostearate are each present in an amount between 0.5% and 5% w/w.

In one example, the medium chain triglycerides comprise caprylic/capric triglycerides in an amount between 60% and 90% w/w, and the glyceryl distearate and glyceryl monostearate are each present in an amount between 0.5% and 5% w/w. The naltrexone may be present in an amount between 3 mg/mL and 15 mg/mL. The phosphatidylcholine may be present in an amount between about 1.5% and about 4% w/w and the lysophosphatidylcholine is present in an amount between about 0.28% and about 0.45% w/w. The anhydrous suspension may further include magnesium stearate and microcrystalline cellulose. The anhydrous suspension may further comprises colloidal silicon dioxide and croscarmellose sodium.

In another aspect, a method of treating opioid use disorder (OUD) or alcohol use disorder (AUD) includes administering to an oral cavity of a subject for oral absorption therein the topical composition of the above aspect to treat opioid use disorder (OUD) or alcohol use disorder (AUD).

### BRIEF DESCRIPTION OF THE DRAWINGS

FIG. 1A shows a semaglutide 1 mg/mL suspension in an anhydrous base vehicle, under magnification, according to various embodiments described herein;

FIG. 1B shows the suspension of FIG. 1A further suspended in water, under magnification, according to various embodiments described herein; and

FIG. 1C shows a 1:1 dilution (water) of a green fluorescent protein 0.1 mg/mL and anhydrous base suspension under fluorescent microscopy according to various embodiments described herein.

### DESCRIPTION

The present disclosure describes various embodiments of a composition for topical administration comprising one or more glucagon-like peptide-1 (GLP-1) receptor agonists, PDE5 inhibitors, selective serotonin receptor agonists, naltrexone, and combinations thereof and methods of making and using the same.

In some embodiments, the topical composition includes one or more GLP-1 receptor agonists. In one example, the GLP-1 receptor agonist may comprise or consist of semaglutide. Additionally or alternatively, the GLP-1 receptor agonist may comprise or consist of liraglutide, exenatide, lixisenatide, taspoglutide, lotiglipron, dulaglutide, tirzepatide, albiglutide, danuglipron, orforglipron, efpeglenatide, amycretin, or combination thereof.

Topical compositions including GLP-1 receptor agonists may be administered as a treatment to lower blood glucose with individuals with type-2 diabetes, to promote weight loss in adjunct with diet and physical activity, or other overindulgence behaviors, such as those described herein. GLP-1 receptor is a transmembrane receptor involved in mediation of intracellular signaling pathways that regulate glucose metabolism, insulin production and secretion, glucagon secretion, gastric emptying, appetited satiation, among others. GLP-1 hormone is a ligand of the GPL-1 receptor and is released in the gastrointestinal tract by “L” cells in response to nutrient consumption. Activation of the GLP-1 receptor upon ligand binding stimulates insulin secretion and slows glucagon secretion, which reduces blood glucose. Activation also slows stomach emptying and interacts with appetite control mechanisms in the brain to signal satiation

In some embodiments, the topical composition comprises one or more PDE5 inhibitors. Example PDE5 inhibitors for use in the topical composition may comprise or consist of vardenafil, tadalafil, or both. Additionally or alternatively, the topical composition may include one or more PDE5 inhibitors comprising sildenafil, avanafil, tadalafil, vardenafil, or combination thereof.

Topical compositions including PDE5 inhibitors may be used to treat erectile dysfunction. Erectile dysfunction is marked by inability to get or keep an erection. PDE5 inhibitors work by increasing blood flow to the penis during sexual stimulation to aid in the erection process. PDE5 inhibitors may also be used to treat symptoms of benign prostate hyperplasia or enlarged prostate such as frequent or urgent need to urinate, difficulty or pain during urination. PDE5 inhibitors may be used to treat pulmonary arterial hypertension (PAH) by relaxing blood vessels in lungs to allow increased blood flow.

Selective serotonin receptor agonists specifically activate serotonin receptors in the brain, thereby mimicking the effects of serotonin (5-HT). In some embodiments, the topical composition includes one or more selective serotonin receptor agonists. Topical compositions include selective serotonin receptor agonists may be used to treatment of migraines and other disorders related to serotonin dysregulation. Example selective serotonin receptor agonists for use in the topical compositions may include triptans. Triptans target the 5-HT1B and 5-HTID receptors. These receptors are located on smooth muscle cells in blood vessels and play a crucial role in vasoconstriction, which helps alleviate migraine symptoms. Triptans work by constricting intracranial blood vessels to reduce the dilation associated with migraine pain and inhibiting release of vasoactive peptides to mitigate further inflammation and pain signaling in the nervous system. Example triptans for use in the topical compositions may comprise or consist of sumatriptan. Additionally or alternatively, the topical composition may include one or more triptans comprising or consisting of almotriptan, eletriptan, frovatriptan, naratriptan, rizatriptan, sumatriptan, zolmitriptan, or combination thereof. Additionally or alternatively, selective serotonin receptor agonists may include compounds that target the 5-HT1F receptor such as Lasmiditan, selective serotonin receptor agonists that target the 5-HT1A receptor to treat hypoactive sexual desire disorder, such as flibanserin.

Topical compositions including naltrexone may be formulated for administration as a non-addictive treatment option for medication-assisted treatment (MAT) to treat both opioid use disorder (OUD) and alcohol use disorder (AUD). Naltrexone binds and blocks opioid receptors, thereby blocking the euphoric and sedative effects of drugs such as heroin, morphine, and codeine, and is reported to reduce opioid cravings, without risk of abuse. Naltrexone is typically administered by pill, intramuscular injection, or as a compounded capsule for a patient to swallow and to be absorbed in the stomach.

In various embodiments, the topical composition may be formulated for topical administration via transmucosal or transdermal routes. For example, the topical composition may be formulated for topical administration to an external skin or mucosal surface. In some examples, the topical composition may be formulated for topical administration to mucosa lining at a natural body orifice, such as the oral cavity, nasal cavity, vagina, or rectum. In one embodiment, the topical composition comprises an anhydrous suspension formulated to form a self-emulsifying liposome formulation when administered or placed in an aqueous environment.

As introduced above, the topical composition may be formulated for topical administration to mucosal tissue for transmucosal delivery. For example, the topical composition may be topically administered to an oral cavity of a subject for oral absorption therein. As a further example, the topical composition may be administered to the oral cavity for oral absorption sublingually, buccally, or otherwise.

When certain active agents are administered via the oral route to the digestive system, plasma albumin binding may be in excess of 99%. Topical administration, e.g., transmucosal via oral absorption, substantially lowers dosing required to achieve a same or similar impact compared to the oral route by nullifying or minimizing the impact of plasma albumin binding and 1st pass metabolism. Thus, the topical compositions and methods for topical administration described herein may beneficially avoid plasma albumin binding and effects of 1st pass metabolism. This breakthrough will reduce dosing requirements and associated patient costs without limiting efficacy. The topical compositions and methods for topical administration described herein may also be used to benefit patients in a manner similar to injectables but without the hassle, pain, and costs of dealing with injectables.

In various embodiments, the topical composition comprising one or more GLP-1 receptor agonists may be used to treat type-2-diabetes or weight loss. In some embodiments, the topical composition may also be used to treat behaviors associated with overindulgence, such as binge behaviors and/or compulsions. Example conditions include those related to binge eating, drinking, or shopping; excessive or compulsive use of social media, electronic devices, television, drugs, tobacco, nicotine, shoplifting; or sex addiction, to name a few. In one example, the topical composition may be used to treat alcoholism.

In various embodiments, the topical composition comprises one or more active agents comprising or consisting of one or more GLP-1 receptor agonists, PDE5 inhibitors, selective serotonin receptor agonist, naltrexone, or combination thereof in a base vehicle for topical oral absorption within the oral cavity. GLP-1 receptor agonists may be selected from semaglutide, liraglutide, exenatide, lixisenatide, taspoglutide, lotiglipron, dulaglutide, tirzepatide, albiglutide, danuglipron, orforglipron, efpeglenatide, amycretin, or combination thereof. PDE5 inhibitors may be selected from vardenafil, tadalafil, sildenafil, avanafil, or combination thereof. The selective serotonin receptor agonist may be selective from, In one example, selective serotonin receptor agonists may be selected from triptans such as one or more of almotriptan, eletriptan, frovatriptan, naratriptan, rizatriptan, sumatriptan, zolmitriptan, or combination thereof. The composition formulated for oral absorption may comprise a solution, semi-solid, or solid format. The topical composition may comprise a powder, solution, suspension, gel, paste, tablet, or lozenge format for transmucosal delivery via oral absorption. The topical composition may be a dry powder, aqueous, a colloidal suspension (e.g., an emulsion), anhydrous, or as otherwise formulated. For example, the topical composition may be provided in an aqueous solution or anhydrous solution base vehicle. The topical composition may be provided in colloid format, such as an emulsion or other suspension format. The topical composition may be provided in a gel format. The topical composition may be provided in a solid format.

The topical composition may be compounded into various oral absorption formats. For example, in one embodiment, the topical composition may be a solution or suspension for application under the tongue or within the buccal pouch by a syringe or dropper. In one example, the topical composition comprises a mouthwash or rinse for transmucosal delivery via the mouth. In some embodiments, the topical composition may be formulated for oral absorption to include flavoring to improve patient compliance due to its ease of use and enhanced taste. In one embodiment, the topical composition comprises a suspension including one or more active agents comprising GLP-1 receptor agonists, PDE5 inhibitors, selective serotonin receptor agonists, naltrexones, or combination thereof in a base vehicle for topical oral absorption. In one example, the base vehicle comprises an anhydrous base vehicle. The anhydrous base vehicle may be configured to self-emulsify when the topical composition is administered to an aqueous environment of the mouth. The suspension may be absorbed in the mouth, e.g., sublingually, buccally, or otherwise. In one example, the suspension is an anhydrous suspension. The suspension may include phospholipids to create an innovative system that delivers the active ingredients directly to the mucous membranes under the tongue and/or buccal pouch. The phospholipids may include lipid aggregates comprising liposomes, micelles, or both.

In various embodiments, the topical composition comprises the active agent encapsulated by, associated with, or in the presence of a liposomal or mixed micelle system during administration. The system may be a mixed micelle system including liposomes or liposome free. The system may include substantially pure liposomes such that the system includes aggregate lipids that are mainly or substantially associated in bilayers. All or a portion of an active agent may be encapsulated by or associated with liposomes, micelles or both. In one example, one or more active agents may not necessarily be encapsulated in liposomes and/or micelles and may be within the topical composition in the presence of lipids such as phospholipids, which may include liposomes and/or mixed micelles. The topical composition may be aqueous, a colloidal suspension (e.g., an emulsion), anhydrous, or as otherwise formulated. In one example, the topical composition comprises a lipid based, preliposomal, or mixed micelle system in an anhydrous suspension base vehicle. The anhydrous suspension base vehicle may ensure the stability and potency of the liposomes or mixed micelle for optimal performance when activated in an aqueous environment. In further embodiments, the liposomal or mixed micelle system in an anhydrous suspension base vehicle. The anhydrous suspension base vehicle may ensure the stability and potency of the liposomes for optimal performance. In some examples, all or a portion of the active agent is solubilized in the anhydrous suspension. The topical composition may have surprising stability. For example, the topical composition may be stable for 90 days or longer. In various embodiments, the topical composition is anhydrous. In further embodiments, the liposomal or mixed micelle system may be utilized in compositions for transmucosal or transdermal delivery at other body surfaces, e.g., external skin, nasal cavity, vaginal, or rectal. In one embodiment, the topical composition includes polycarbophil to prolong contact. The topical composition may be anhydrous and activate upon interaction with an aqueous environment to self-emulsify and form an active composition comprising a liposomal or mixed micelle system. In an embodiment for oral absorption, the topical composition employs film-forming adhesion ingredients that create a protective film to minimize initial contact of the active agent drug with tastebuds. In one example, the topical compositions, e.g., the anhydrous suspension base vehicle thereof, includes polycarbophil, glyceryl distearate, and glyceryl monostearate, or equivalents, formulated to form a protective matrix/film that adheres to the mucosa to prolong contact while also minimizing initial drug contact with tastebuds to reduce bitterness.

The topical composition may be provided at any suitable dosage of active agent, which may be determined by those skilled in the art in view of the present disclosure. In various applications, choice of dosage may consider one or more of the conditions treated, severity, patient profile, dosing frequency, format, or route of delivery. For example, with respect to GLP-1 receptor agonists, satiation treatment for weight loss, for instance, may benefit from larger doses than doses for diabetes. Overindulgence treatment may benefit from larger doses than satiation. Patients with greater body mass may benefit from larger doses than those with less body mass. Transdermal delivery may benefit from larger doses than transmucosal delivery. Various dosing schedule may be used. For example, dosing may be once or twice a day or as otherwise prescribed. Dosing may be weekly or twice weekly in some applications. Larger dosages may be found appropriate in less frequent dosing regimens.

In various embodiments, the topical composition is administered in an active agent dose between 0.01 mg to 100 mg for oral absorption or other transmucosal or transdermal route. For example, the topical composition may be administered in a dose between about 1 mg and about 30 mg, between about 20 mg and about 40 mg, between about 30 mg and about 50 mg, between about 40 mg and about 60 mg, between about 50 mg and about 100 mg, between about 80 mg and about 100 mg, between about 60 mg and about 100 mg, between about 10 mg and about 40 mg, between about 20 mg and about 60 mg, between about 30 mg and about 70 mg, between about 40 mg and about 80 mg, between about 50 mg and about 90 mg, between about 60 mg and about 100 mg, between about 70 mg and about 100 mg, greater than 10 mg, greater than 25 mg, greater than 50 mg, or greater than 75 mg. In one example, the above dosing is once or twice a day, split between multiple doses a day, every other day, twice weekly, or as otherwise determined suitable.

In one embodiment, the topical composition may be administered to the mouth for oral absorption or other transmucosal delivery route in an active agent dose amount between about 0.1 mg and about 1 mg, between about 0.1 mg and about 0.8 mg, between about 0.1 mg and about 0.6 mg, between about 0.1 mg and about 0.6 mg, between about 0.25 mg and about 0.5 mg, or between about 0.5 mg and about 1 mg. In some embodiments, dosing with respect to some GLP-1 receptor agonists, such as exenatide or lixisenatide, may be less than 0.01 mg. For example, dosing may be between about 0.001 mg and 0.01 mg, such as between about 0.003 mg and about 0.01 mg, between about 0.005 mg and about 0.01 mg. Such dosing may be beneficial in daily or twice daily dosing via transmucosal delivery. Larger dosing may be determined to be appropriate for transmucosal or transdermal delivery. For example, the topical composition may be administered in a dose between about 1 mg and 20 mg, such as between 1 mg and about 5 mg, between about 1 mg and about 10 mg, between about 1 mg and about 15 mg, between about 2 mg and about 6 mg, between about 3 mg and about 10 mg, between about 4 mg and about 15 mg, between about 6 mg and about 15 mg, between about 10 mg and about 20 mg, between about 10 mg and about 15 mg, or between about 15 mg and about 20 mg.

In various embodiments, the topical composition may comprise semaglutide and be formulated to be administered to the mouth for oral absorption or other transmucosal delivery route. The topical composition may be administered in dosages comprising an amount of semaglutide between about 0.1 mg and about 15 mg, which includes any interval therebetween, such as between about 0.1 mg and about 10 mg, between about 0.1 mg and about 7 mg, between about 0.1 mg and about 5 mg, between about 0.5 mg and about 10 mg, between about 0.5 mg and about 5 mg, between about 0.5 mg and about 3 mg, about 0.5 mg and about 1.5 mg, between about 1 mg and about 15 mg, between about 1 mg and about 10 mg, between about 1 mg and about 5 mg, between about 1 mg and about 4 mg, between about 1 mg and about 3 mg, between about 1 mg and about 2.5 mg, between about 1 mg and about 2 mg, between about 2.5 mg and about 10 mg, between about 2.5 mg and about 5 mg, between about 2.5 mg and about 4 mg, between about 3.5 mg and about 15 mg, between about 3.5 mg and about 10 mg, between about 3.5 mg and about 5 mg, between about 4 mg and about 5 mg, between about 5 mg and about 15 mg, between about 5 mg and about 10 mg, between about 5 mg and about 7.5 mg, between about 7.5 mg and about 15 mg, between about 7.5 mg and about 10 mg, between about 10 mg and about 15 mg, or between about 12.5 mg and about 15 mg. In one embodiment, the topical composition is administered in a dosage of less than about 15 mg, less than about 12.5 mg, less than about 10 mg, less than about 7.5 mg, less than about 5 mg, less than about 4 mg, less than about 3 mg, less than about 2 mg, less than about 1 mg, greater than about 12.5 mg, greater than about 10 mg, greater than about 7.5 mg, greater than about 5 mg, greater than about 4 mg, greater than about 3 mg, greater than about 2 mg, greater than about 1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6.5 mg, about 7 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, or about 15 mg semaglutide. Typical dosages may include between about 1 mg and about 4 mg of semaglutide. Semaglutide may typically be administered once daily or as otherwise deemed medically suitable.

In various embodiments, the topical composition may comprise a PDE5 inhibitor and be formulated to be administered to the mouth for oral absorption or other transmucosal delivery route. The topical composition may be administered in dosages comprising an amount of PDE5 inhibitor between about 0.1 mg and about 50 mg, which includes any interval therebetween. In one example, the PDE5 inhibitor comprises tadalafil and is administered in an amount to deliver between about 0.1 mg and about 15 mg, about 0.1 mg and about 10 mg, between about 0.1 mg and about 7 mg, between about 0.1 mg and about 5 mg, between about 0.5 mg and about 10 mg, between about 0.5 mg and about 5 mg, between about 0.5 mg and about 3 mg, about 0.5 mg and about 1.5 mg, between about 1 mg and about 15 mg, between about 1 mg and about 10 mg, between about 1 mg and about 5 mg, between about 1 mg and about 4 mg, between about 1 mg and about 3 mg, between about 1 mg and about 2.5 mg, between about 1 mg and about 2 mg, between about 2.5 mg and about 10 mg, between about 2.5 mg and about 5 mg, between about 2.5 mg and about 4 mg, between about 3.5 mg and about 15 mg, between about 3.5 mg and about 10 mg, between about 3.5 mg and about 5 mg, between about 4 mg and about 5 mg, between about 5 mg and about 15 mg, between about 5 mg and about 10 mg, between about 5 mg and about 7.5 mg, between about 7.5 mg and about 15 mg, between about 7.5 mg and about 10 mg, between about 10 mg and about 15 mg, or between about 12.5 mg and about 15 mg tadalafil. In one embodiment, the topical composition is administered in a dosage of less than about 15 mg, less than about 12.5 mg, less than about 10 mg, less than about 7.5 mg, less than about 5 mg, less than about 4 mg, less than about 3 mg, less than about 2 mg, less than about 1 mg, greater than about 12.5 mg, greater than about 10 mg, greater than about 7.5 mg, greater than about 5 mg, greater than about 4 mg, greater than about 3 mg, greater than about 2 mg, greater than about 1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6.5 mg, about 7 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, or about 15 mg tadalafil. Typical dosages of tadalafil may include between about 1.5 mg and about 4 mg. In some embodiments, tadalafil may typically be administered once daily or as otherwise deemed medically suitable.

In one example, the PDE5 inhibitor comprises vardenafil and is administered in an amount to deliver between about 0.5 mg and about 35 mg, between about 0.5 and about 30 mg, between about 1 mg and about 25 mg, between about 1 mg and about 20 mg, between about 1 mg and about 15 mg, between about 1 mg and about 10 mg, between about 1 mg and about 5 mg, between about 1 mg and about 4 mg, between about 1 mg and about 3 mg, between about 1 mg and about 2.5 mg, between about 1 mg and about 2 mg, between about 3.5 mg and about 30 mg, between about 3.5 mg and about 25 mg, between about 3.5 mg and about 20 mg, between about 5 mg and about 40 mg, between about 5 mg and about 35 mg, between about 5 mg and about 30 mg, between about 5 mg and about 25 mg, between about 5 mg and about 20 mg, between about 5 mg and about 15 mg, between about 5 mg and about 10 mg, between about 7.5 mg and about 22.5 mg, between about 7.5 mg and about 15 mg, between about 7.5 mg and about 10 mg, between about 10 mg and about 40 mg, between about 10 mg and about 35 mg, between about 10 mg and about 30 mg, between about 10 mg and about 25 mg, between about 10 mg and about 20 mg, between about 10 mg and about 15 mg, between about 12.5 mg and about 30 mg, between about 12.5 mg and about 25 mg, between about 12.5 mg and about 20 mg, between about 12.5 mg and about 15 mg, between about 15 mg and about 30 mg, between about 15 mg and about 25 mg, or between about 15 mg and about 20 mg vardenafil. In one embodiment, the topical composition is administered in a dosage of less than about 40 mg, less than about 37.5 mg, less than about 35 mg, less than about 32.5 mg, less than about 30 mg, less than about 27.5 mg, less than about 25 mg, less than about 22.5 mg, less than about 20 mg, less than about 17.5 mg, less than about 15 mg, less than about 12.5 mg, less than about 10 mg, less than about 7.5 mg, less than about 5 mg, less than about 4 mg, less than about 3 mg, less than about 2 mg, less than about 1 mg, greater than about 37.5 mg, greater than about 35 mg, greater than about 32.5 mg, greater than about 30 mg, greater than about 27.5 mg, greater than about 25 mg, greater than about 22.5 mg, greater than about 20 mg, greater than about 17.5 mg, greater than about 15 mg, greater than about 12.5 mg, greater than about 10 mg, greater than about 7.5 mg, greater than about 5 mg, greater than about 4 mg, greater than about 3 mg, greater than about 2 mg, greater than about 1 mg, about 1.5 mg, about 2 mg, about 2.5 mg, about 3 mg, about 3.5 mg, about 4 mg, about 4.5 mg, about 5 mg, about 5.5 mg, about 6.5 mg, about 7 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, or about 30 mg vardenafil. Typical dosages of vardenafil may include between about 5 mg and about 20 mg. In some embodiments, vardenafil may typically be administered once daily or as otherwise deemed medically suitable.

In various embodiments, the topical composition may comprise a naltrexone and be formulated to be administered to the mouth for oral absorption or other transmucosal delivery route. The topical composition may be administered in dosages comprising an amount of naltrexone between about 0.1 mg and about 50 mg, which includes any interval therebetween, such as between about 0.1 mg and about 15 mg, about 0.1 mg and about 10 mg, between about 0.1 mg and about 7 mg, between about 0.1 mg and about 5 mg, between about 0.5 mg and about 10 mg, between about 0.5 mg and about 5 mg, between about 0.5 mg and about 3 mg, about 0.5 mg and about 1.5 mg, between about 1 mg and about 15 mg, between about 1 mg and about 10 mg, between about 1 mg and about 5 mg, between about 1 mg and about 4 mg, between about 1 mg and about 3 mg, between about 1 mg and about 2.5 mg, between about 1 mg and about 2 mg, between about 2.5 mg and about 10 mg, between about 2.5 mg and about 5 mg, between about 2.5 mg and about 4 mg, between about 3.5 mg and about 15 mg, between about 3.5 mg and about 10 mg, between about 3.5 mg and about 5 mg, between about 4 mg and about 5 mg, between about 5 mg and about 15 mg, between about 5 mg and about 10 mg, between about 5 mg and about 7.5 mg, between about 7.5 mg and about 15 mg, between about 7.5 mg and about 10 mg, between about 10 mg and about 15 mg, or between about 12.5 mg and about 15 mg. In one embodiment, the topical composition is administered in a dosage of less than about 15 mg, less than about 12.5 mg, less than about 10 mg, less than about 7.5 mg, less than about 5 mg, less than about 4 mg, less than about 3 mg, less than about 2 mg, less than about 1 mg, greater than about 12.5 mg, greater than about 10 mg, greater than about 7.5 mg, greater than about 5 mg, greater than about 4 mg, greater than about 3 mg, greater than about 2 mg, greater than about 1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6.5 mg, about 7 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, or about 15 mg naltrexone. Typical dosages of naltrexone may include between about 1 mg and about 5 mg. In some embodiments, naltrexone may typically be administered once daily or as otherwise deemed medically suitable.

In various embodiments, the topical composition may comprise a selective serotonin receptor agonist and be formulated to be administered to the mouth for oral absorption or other transmucosal delivery route. The topical composition may be administered in dosages comprising an amount of selective serotonin receptor agonist between about 0.1 mg and about 50 mg, which includes any interval therebetween. In one embodiment, the selective serotonin receptor agonist comprises or consists of a triptan. In one example, the triptan comprises sumatriptan and is administered in an amount to deliver between about 0.1 mg and about 45 mg, between about 0.5 mg and about 40 mg, between about 0.5 mg and about 35 mg, between about 0.5 mg and about 30 mg, about 0.5 mg and about 22.5 mg, between about 2 mg and about 40 mg, between about 2 mg and about 35 mg, between about 2 mg and about 30 mg, between about 2 mg and about 25 mg, between about 2 mg and about 20 mg, between about 2 mg and about 25 mg, between about 5 mg and about 40 mg, between about 5 mg and about 35 mg, between about 5 mg and about 30 mg, between about 5 mg and about 25 mg, between about 5 mg and about 20 mg, between about 5 mg and about 15 mg, between about 5 mg and about 10 mg, between about 10 mg and about 40 mg, between about 10 mg and about 35 mg, between about 10 mg and about 30 mg, between about 10 mg and about 25 mg, between about 10 mg and about 20 mg, between about 10 mg and about 15 mg, between about 15 mg and about 45 mg, between about 15 mg and about 40 mg, between about 15 mg and about 35 mg, between about 15 mg and about 30 mg, between about 15 mg and about 25 mg, or between about 15 mg and about 20 mg sumatriptan. In one embodiment, the topical composition is administered in a dosage of less than about 45 mg, less than about 40 mg, less than about 37.5 mg, less than about 35 mg, less than about 32.5 mg, less than about 30 mg, less than about 27.5 mg, less than about 25 mg, less than about 22.5 mg, less than about 20 mg, less than about 17.5 mg, less than about 15 mg, less than about 12.5 mg, less than about 10 mg, less than about 7.5 mg, less than about 5 mg, less than about 4 mg, less than about 3 mg, less than about 2 mg, less than about 1 mg, greater than about 40 mg, greater than about 37.5 mg, greater than about 35 mg, greater than about 32.5 mg, greater than about 30 mg, greater than about 27.5 mg, greater than about 25 mg, greater than about 22.5 mg, greater than about 20 mg, greater than about 17.5 mg, greater than about 15 mg, greater than about 12.5 mg, greater than about 10 mg, greater than about 7.5 mg, greater than about 5 mg, greater than about 4 mg, greater than about 3 mg, greater than about 2 mg, greater than about 1 mg, about 5 mg, about 10 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg sumatriptan. Typical dosages of sumatriptan may include between about 15 mg and about 30 mg. In some embodiments, sumatriptan may typically be administered at the onset of a migraine and, if needed, repeated thereafter, such as an hour after the initial dose or as otherwise deemed medically suitable.

In various embodiments, the topical composition may comprise amycretin and be formulated to be administered to the mouth for oral absorption or other transmucosal delivery route. The topical composition may be administered in dosages comprising an amount of amycretin between about 0.1 mg and about 50 mg, which includes any interval therebetween, such as between about 0.1 mg and about 30 mg, between about 0.1 mg and about 20 mg, between about 0.1 mg and about 15 mg, between about 0.5 mg and about 10 mg, between about 0.5 mg and about 5 mg, between about 0.5 mg and about 3 mg, about 0.5 mg and about 1.5 mg, between about 1 mg and about 15 mg, between about 1 mg and about 10 mg, between about 1 mg and about 5 mg, between about 1 mg and about 4 mg, between about 1 mg and about 3 mg, between about 1 mg and about 2.5 mg, between about 1 mg and about 2 mg, between about 2.5 mg and about 10 mg, between about 2.5 mg and about 5 mg, between about 2.5 mg and about 4 mg, between about 3.5 mg and about 15 mg, between about 3.5 mg and about 10 mg, between about 3.5 mg and about 5 mg, between about 4 mg and about 5 mg, between about 5 mg and about 15 mg, between about 5 mg and about 10 mg, between about 5 mg and about 7.5 mg, between about 7.5 mg and about 15 mg, between about 7.5 mg and about 10 mg, between about 10 mg and about 15 mg, between about 15 mg and about 45 mg, or between about 12.5 mg and about 15 mg. In one embodiment, the topical composition is administered in a dosage of less than about 15 mg, less than about 12.5 mg, less than about 10 mg, less than about 7.5 mg, less than about 5 mg, less than about 4 mg, less than about 3 mg, less than about 2 mg, less than about 1 mg, greater than about 40 mg, greater than about 30 mg, greater than about 20 mg, greater than about 15 mg, greater than about 12.5 mg, greater than about 10 mg, greater than about 7.5 mg, greater than about 5 mg, greater than about 4 mg, greater than about 3 mg, greater than about 2 mg, greater than about 1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6.5 mg, about 7 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 20 mg, about 25 mg, about 30 mg, about 35 mg, about 40 mg, about 45 mg, or about 50 mg amycretin. Amycretin may typically be administered once daily or as otherwise deemed medically suitable.

In some embodiments, the composition includes multiple of the active agents and is administered in dosages including the above amounts of the respective actives. For example, the topical composition may include a PDE5 inhibitor, such as tadalafil or vardenafil, and a GLP-1 receptor agonist, such as semaglutide, wherein the topical composition is administered such that the active agents are present in the above dosages.

In some embodiments, the topical composition is administered in dosages comprising between about or more typically between about 1 mg and about 4 mg. an amount between about 0.25

The topical composition may be provided at any suitable concentration. Appropriate concentrations may be determined by those skilled in the art in view of the present disclosure. In various applications, choice of concentration may consider the desired dosage, administration volume/weight, route of delivery, or other consideration. For example, higher concentrations may be reasonable for lower administration volumes. In one example, concentration of GLP-1 receptor agonist, such as semaglutide, in the topical composition may be between about 0.001% and about 10% (w/v). For example, the topical composition formulated for transmucosal delivery may be provided at a concentration between about 0.001% and about 0.01%, between about 0.001% and about 0.1%, about 0.05% and about 1%, between about 0.05% and about 0.5%, between about 0.08% and about 0.2%, between about 0.08% and about 0.15%, between about 0.075% and about 0.125%, between about 0.01% and about 0.1%, between about 0.1% and about 1%, between about 1% and about 2%, between about 2% and about 5%, between about 4% and about 8%, greater than about 0.08%, less than about 0.5%, less than about 0.3%, or less than about 1.5%. In some embodiments, the topical composition may be formulated to include a selective serotonin receptor agonist at a concentration between about 0.5% and about 25%, such as between about 0.5% and about 1.5%, about 0.5% and about 5%, between about 0.5% and about 10%, between about 1.5% and about 5%, between about 1% and about 15%, between about 2% and about 5%, between about 2% and about 10%, between about 2% and about 15%, between about 5% and about 10%, between about 5% and about 15%, between about 10% and about 25%, greater than about 1%, less than about 3%, less than about 5%, or less than about 10%. In one example, the topical composition includes sumatriptan at a concentration between about 5 mg/mL and about 50 mg/ml, between about 10 mg/mL and about 40 mg/mL, between about 15 mg/mL and about 35 mg/mL, less than about 40 mg/mL, less than about 30 mg/mL, less than about 25 mg/mL, greater than about 5 mg/mL, greater than about 10 mg/mL, greater than about 15 mg/mL, greater than about 20 mg/mL, greater than about 25 mg/mL, or about 25 mg/mL. In some embodiments, the topical composition may be formulated to includes a PDE5 inhibitor at a concentration between about 0.5% and about 25%, such as between about 0.5% and about 1.5%, about 0.5% and about 5%, between about 0.5% and about 10%, between about 1.5% and about 5%, between about 1% and about 15%, between about 2% and about 5%, between about 2% and about 10%, between about 2% and about 15%, between about 5% and about 10%, between about 5% and about 15%, between about 10% and about 25%, greater than about 1%, less than about 3%, less than about 5%, or less than about 15%. In one example, the topical composition includes tadalafil at a concentration between about 0.5 mg/mL and about 10 mg/mL, or between about 1 mg/mL and about 5 mg/mL, or greater than about 1 mg/mL, greater than about 1.5 mg/mL, greater than about 2 mg/mL, less than about 5 mg/mL, less than about 3 mg/mL, or about 2.5 mg/mL. In one example, the topical composition includes vardenafil at a concentration between about 2 mg/mL and about 20 mg/mL, or between about 5 mg/mL and about 15 mg/mL, or greater than about 1 mg/mL, greater than about 2.5 mg/mL, greater than about 5 mg/mL, greater than about 7.5 mg/mL, greater than about 10 mg/mL, less than about 15 mg/mL, less than about 12 mg/mL, or about 10 mg/mL. In some embodiments, the topical composition may be formulated to naltrexone at a concentration between about 0.5% and about 35%, such as between about 1% and about 30%, about 1% and about 25%, between about 3% and about 20%, between about 3% and about 15%, between about 5% and about 10%, between about 6% and about 9%, greater than about 1%, greater than about 3%, greater than about 6%, greater than about 8%, greater than about 12%, less than about 25%, less than about 20%, less than about 15%, less than about 10%, about 9%, or about 6%. Greater or lesser concentrations than those listed above may also be used. Compositions for transdermal delivery may be provided at concentrations similar to those listed above with respect to transmucosal. In one embodiment, the topical composition comprises suspension provided in active agent concentrations of at about 0.5 mg/mL, about 1 mg/mL, about 1.5 mg/mL, about 2 mg/mL, about 2.5 mg/mL, about 3 mg/mL, about 4 mg/mL, about 6 mg/mL, about 9 mg/mL, about 10 mg/mL, about 25 mg/mL, about 50 mg/mL, or about 100 mg/mL.

In one formulation, the topical composition may comprise a dry powder format. The powder may be loose, encapsulated, or compressed in a tablet form for administration to the mouth, e.g., a buccal or sublingual tablet. The powder may be placed under the tongue, along the gums and/or cheeks (e.g., within the buccal pouch) to dissolve, wet, hydrate, solubilize, or reconstitute all or a portion of the powder contents with saliva in situ for transmucosal absorption. For example, in a preliposome format, the powder may reconstitute in situ for liposome formation and delivery by oral absorption. In one example, the powder may reconstitute and self-emulsify into a liposome or mix micelle system. In one embodiment, the powder may be provided in a pouch for administration to the mouth. In one embodiment, the powder is provided within a capsule including a liquid separated from the powder. The capsule may be mechanically broken down or dissolved in biological fluid to combine the powder and liquid in situ. In various embodiments, the topical composition comprises a powder base vehicle. In these or other embodiments, the topical composition comprises one or more surfactants, pH adjusting agents, flavorants, viscosity modifiers, preservatives, or the like.

In various embodiments, the powder may comprise or consist of particle sizes from very fine to micronized, such as less than about 50 microns, less than about 40 microns, less than about 30 microns, less than about 25 microns, or less than about 20 microns. In some examples, the particle sizes represent an average or a majority of the particles within the range, such as at least 50%, at least 60%, at least 70%, at least 80%, at least 90%, at least 95%, or at least 98%. In a further example, the remaining particles outside the particle range may be no more than about 25%, about 20%, about 15%, about 10%, or about 5% larger.

As described in more detail below, in some embodiments, compositions in a powder format may be topically administered to skin, mucosa, or both. In further embodiments, the topical composition may be compounded from a powder or powder mixture that is mixed with a base vehicle. The base vehicle may comprise a solution (e.g., aqueous, anhydrous), colloidal suspension (e.g., emulsion, anhydrous suspension), oil, ointment, cream, lotion, paste, gel, or other suitable base vehicle for topical administration.

The topical composition comprising a powder for administration in a powder format or a powder for mixing with a non-powder base vehicle, may include one or more additional ingredients, such as xylitol, poloxamers, salcaprozate sodium, sodium caprate, lactose, starch, magnesium stearate, cellulose or cellulose derivatives, microcrystalline cellulose, sugar, sugar alcohol, povidone, talc, or combination thereof. In embodiments comprising or formulated for combination with a liquid or semi-liquid base vehicle, the powder, base vehicle, or otherwise the topical composition may include one or more additional ingredients, such as xylitol, poloxamers, salcaprozate sodium, sodium caprate, lactose, starch, magnesium stearate, cellulose or cellulose derivatives, microcrystalline cellulose, sugar, sugar alcohol, povidone, talc, or combination thereof. In some embodiments, additional ingredients in the topical composition may include a sodium glucose cotransporter 2 inhibitor. For example, a sodium glucose cotransporter 2 inhibitor may be selected from one or more of canagliflozin, dapagliflozin, empagliflozin or ertugliflozin.

As introduced above, in some embodiments, the topical composition comprises a liposomal or mixed micelle delivery system. In one example, the topical composition comprises a powder mixture of preliposomes and active agent. In a further example, the preliposomal powder mixture may include one or more additional ingredients, such as xylitol, poloxamers, salcaprozate sodium, sodium caprate, lactose, starch, magnesium stearate, cellulose or cellulose derivatives, microcrystalline cellulose, sugar, sugar alcohol, povidone, talc, or combination thereof. The preliposomal powder may include or be mixed with active agent in a powder or liquid form. The preliposomal powder may be reconstituted prior to administration. For example, the preliposomal powder may be formulated for reconstitution with a liquid medium to compound a solution or suspension. The liquid medium may comprise, for example, an aqueous medium, anhydrous medium, emulsion, oil, water, or other suitable medium for topical administration to the mouth or other desired body surface. In one example, the preliposomal powder may include or be mixed with active agent in a powder or liquid format. The mixture may be further mixed with an anhydrous medium prior to administration, which may be minutes, hours, days, or months prior to administration.

In some embodiments, the preliposomal powder may be formulated for reconstitution in situ. For example, the topical composition powder may be administered to a skin or mucosal surface, such as the mouth, for reconstitution upon interaction with body fluids. For instance, the topical composition may comprise a powder for administration to the nasal cavity. Administration may deposit the topical composition along nasal mucosal of the nares. Administration may deposit the topical composition along mucosa of the sinus. In one example, the topical composition comprises preliposomes configured for liposomal delivery upon reconstitution with body fluid as described herein. As introduced above, powder formats may include tablets, capsules, or loose powder. In one example, the preliposome powder may be dispersed within an oral lozenge medium, buccal tablet, sublingual tablet, or contained within a pouch for oral absorption upon reconstitution in situ. In some embodiments, the powder or base vehicle medium may include one or more flavorants and/or sweeteners described herein. In some embodiments, the topical composition comprising a preliposome powder may be mixed with a base vehicle to compound a suspension, cream, lotion, ointment, gel, or paste for topical administration via transmucosal or transdermal delivery.

In various embodiments, the topical composition may be provided in a liquid format. The liquid format may comprise an anhydrous lipid suspension comprising the one or more active agents suspending in a lipid vehicle including phospholipids. Such suspensions may comprise liposomal or mixed micelle systems. In one example, liposomal or mixed micelle systems are referred to as preliposomes formulated to form liposomal or mixed micelle systems upon the addition of aqueous liquid or when placed within an aqueous environment, which may include the cite of administration. In a further example, the preliposomes and base vehicle comprise self-emulsifying liposomes upon addition of aqueous liquid or when placed within an aqueous environment.

In one embodiment, the topical composition comprises a suspension. The suspension may be administered to the mouth for oral absorption. In one example, the suspension may be compounded just before administration by combining ingredients of the topical composition with a liquid base vehicle, which may include one or more components thereof, and mixing to compound the suspension. In another example, the suspension may be compounded days, weeks, or months before administration. Thus, the suspension may be stable over extended periods of time. In one example, the suspension comprises an anhydrous base vehicle. The base vehicle may comprise an oil, which may include an oil mixture. The base vehicle may comprise a lipid-based drug delivery system. In one formulation, the base vehicle comprises an emulsion. The base vehicle may be a buffered suspending base vehicle, syrup, elixir, or the like. The suspension may include various additional ingredients such as surfactants, penetrant enhancers, chelates, pH adjusting agents, flavorants and/or sweeteners, viscosity modifiers, preservatives, release agent (e.g., sustained, controlled, delayed, or extended), absorption modifiers, or the like. In one example, the one or more additional ingredients comprise xylitol, poloxamers, salcaprozate sodium, sodium caprate, lactose, starch, magnesium stearate, cellulose or cellulose derivatives, microcrystalline cellulose, sugar, sugar alcohol, povidone, talc, or combination thereof. In one example, the topical composition does not include preservatives. In one embodiment, the topical composition comprises semaglutide, tadalafil, vardenafil, naltrexone, sumatriptan, or combination thereof suspended in an anhydrous base vehicle including lipids in a preliposome formulation. In various embodiments, the topical composition comprises one or more permeation enhancers to boost delivery into and through buccal or gingival tissue. In one example, the topical composition or the base vehicle comprises diethylene glycol monoethyl ether NF.

As introduced above, in various embodiments, the topical composition comprises a liposomal or mixed micelle system. For example, the topical composition may include a liposomal base vehicle suspension encapsulating or associated with active agents. In a further example, liposomes may encapsulate or otherwise associate with active agents and one or more additional ingredients. The topical composition may comprise mixed micelles. The mixed micelles may encapsulate or otherwise associate with active agents. In a further example, mixed micelles may encapsulate or otherwise associate with active agents and one or more additional ingredients. The one or more additional ingredients in the formulations herein, which may include liquid, solution, suspension, powder, or solid or semi-solid dosage forms may comprise xylitol, poloxamers, salcaprozate sodium, sodium caprate, lactose, starch, magnesium stearate, cellulose or cellulose derivatives, microcrystalline cellulose, sugar, sugar alcohol, povidone, talc, or combination thereof. In a further or another example, one or more additional ingredients may be within the liposome containing medium, which may be in addition to or instead of being encapsulated within the liposomal delivery base vehicle. For example, xylitol, poloxamers, or both may be within the liposome containing medium and active agent may be encapsulated alone or together with one or more additional ingredients selected from salcaprozate sodium, sodium caprate, lactose, starch, magnesium stearate, cellulose or cellulose derivatives, microcrystalline cellulose, sugar, sugar alcohol, povidone, talc, or combination thereof. In some embodiments, additional ingredients may include a sodium glucose cotransporter 2 inhibitor, such as one selected from one or more of canagliflozin, dapagliflozin, empagliflozin or ertugliflozin.

In various embodiments, the liposomal or mixed micelle system comprises liposomes and/or micelles within an anhydrous medium, which may be preliposomal or otherwise lipid based prior to initiation upon aqueous exposure. In another example, the base vehicle comprises liposomes and/or micelles within an emulsion that self-emulsifies when contacted with water to form liposomes and mixed micelles. In one example, the topical composition comprises an anhydrous liposomal or micelle base vehicle suspension encapsulating, associating, or in the presence of active agent. In some embodiments, the suspension is compounded by mixing a preliposomal powder, active agent, and a liquid base vehicle, which may be just prior to administration or earlier. In one embodiment, a liposomal or mixed micelle system comprises an anhydrous liquid base vehicle, e.g., comprising lipids such as phospholipids, oils, or both. The anhydrous liquid base vehicle may have a preliposomal format configured to self-emulsify as described herein. In other formulations, a liquid base vehicle comprises an aqueous base vehicle. In some embodiments, the suspension is compounded by mixing the base vehicle with the one or more active agents. The base vehicle may comprise an anhydrous liquid including phospholipids. The base vehicle may be configured to self-emulsify in an aqueous environment, which may be an in situ environment driven by aqueous biological fluid or aqueous liquid added to or supplemented to an administration site or added prior to administration to the administration site.

In various embodiments, the topical composition may include additional ingredients which may be mixed with or included in the base delivery vehicle, such as an anhydrous self-emulsifying liposomal or mixed micelle delivery base vehicle formulated to self-emulsify in an aqueous environment to form liposomes, mixed micelles, or both. In one example, additional ingredients may include one or more of croscarmellose sodium, lactose anhydrous, lactose monohydrate, magnesium stearate, mannitol, microcrystalline cellulose, talc, titanium dioxide, or triacetin. In one example, additional ingredients may include one or more of carnauba wax, colloidal silicon dioxide, croscarmellose sodium, hypromellose, hydroxypropyl cellulose, lactose anhydrous, magnesium stearate, microcrystalline cellulose, or polyethylene glycol. In one example, additional ingredients may include one or more of croscarmellose sodium, lactose monohydrate, low-substituted hydroxypropyl cellulose, magnesium stearate, microcrystalline cellulose, or sodium lauryl sulphate. In one example, additional ingredients may include one or more of croscarmellose sodium, hydroxypropyl cellulose, hypromellose, lactose monohydrate, magnesium stearate, microcrystalline cellulose, sodium lauryl sulfate, titanium dioxide, or triacetin. In one example, additional ingredients may include one or more of aspartame, colloidal silicon dioxide, crospovidone, magnesium stearate, microcrystalline cellulose, or titanium dioxide. In one example, additional ingredients may include one or more of colloidal silicon dioxide, crospovidone, hydroxypropyl cellulose, microcrystalline cellulose, magnesium stearate, or talc. In one example, such compositions are formulated for oral absorption or otherwise mucosal absorption, such as at a body orifice.

The topical composition comprising a liposomal or mixed micelle system may be formulated for oral absorption, e.g., sublingually, buccally, or otherwise. The topical composition comprising a liposomal or mixed micelle system may be formulated for transmucosal delivery rectally, nasally, or vaginally. The topical composition comprising a liposomal or mixed micelle system may be formulated for transdermal delivery to an exterior skin surface. In some embodiments, the topical composition comprising a liposomal or mixed micelle system comprises a suspension, ointment, cream, lotion, gel, paste, or solution.

In various embodiments, the topical composition includes one or more permeation enhancing ingredients. Permeation enhancing ingredients may be provided within a vehicle or added a vehicle described here. Permeation enhancing ingredients may be included to boost delivery into and through buccal or gingival tissue. Example, permeation enhancing ingredients may include diethylene glycol monoethyl ether NF (Transcutol HP). In some embodiments, permeation enhancing ingredients are selected that also have emulsifying or surfactant action to impart a self-emulsifying aspect to the topical composition. Permeation enhancing ingredients may be included in compositions comprising liposomal or mixed micelle systems.

In various embodiments, the topical composition configured for oral absorption includes one or more flavorants and/or sweeteners. For example, flavorants and/or sweeteners may be provided in a powder composition format (loose powder, pouch, tablet, capsule, or otherwise), suspension composition format, a solution composition format, a lozenge composition format, or other format for oral absorption. The flavorant may be natural, artificial, synthetic, or combination thereof. Sweeteners may comprise one or more of artificial, synthetic, or natural sweeteners. In one example, sweeteners include Siraitia grosvenorii (monk) fruit. In this or another example, sweeteners include sucralose. The flavorant may include methyl salicylate, one or more natural essential oils, or combination thereof. The flavorant may include a natural or artificial oil. Oils may include lemon, peppermint, lavender, or rose, as examples. The flavorant may comprise or be obtained from a flavored powder such as banana, chocolate, strawberry, vanilla, pineapple, mango, or other suitable flavored powder. Example flavorants may include bitter taste flavorants such as chocolate, peanut oil, olive oil, walnut, almond, wild cherry, sesame oil, corn oil, mint, anise, monk fruit, marshmallow oil, or combination thereof. Flavorants may include cherry, grape, raspberry, peppermint, cinnamon, peach, orange, mixed fruit, apricot, honey, butterscotch, clove, ginger, ethyl maltol, cardamon, capric acid, malic acid, ethyl acetate, methionine, maltol, spearmint, menthol, or combination thereof.

In various embodiments, the topical composition formulated for oral absorption may be mixed with water or an aqueous solution such as juice prior to the time of administration. For example, a powder, aqueous suspension, colloidal suspension, or anhydrous suspension as described above and elsewhere herein may be mixed with water or an aqueous solution such as juice prior to administration, which may be immediately prior, e.g., within an hour, or longer for stable formulations. In one example, the topical composition comprising a self-emulsifying anhydrous base vehicle may be configured to be mixed with an aqueous liquid prior to administration. The self-emulsifying anhydrous base may comprise an anhydrous liquid or powder. The self-emulsifying anhydrous base may comprise a lipid based delivery system. The self-emulsifying anhydrous base may comprise a liposomal and/or mixed micelle system formulated to form self-emulsifying liposomes and/or mixed micelle system when in an aqueous environment or upon addition of an aqueous liquid. For example, an anhydrous base vehicle may comprise a liposomal delivery base vehicle formulated to self-emulsify to form a liposomal or mixed micelle system. Such a base vehicle may be referred to as a self-emulsifying anhydrous liposomal or mixed micelle delivery base vehicle. A topical composition including such a delivery vehicle may comprise a self-emulsifying liposome formulation formulated to self-emulsify in an aqueous environment to form a liposomal system, mixed micelle system, or both. Active agents may be encapsulated or associated with liposomes or mixed micelles.

As introduced above, the topical composition may be formulated for transmucosal and/or transdermal delivery via application to external skin or mucosa of the oral cavity, nasal cavity, vagina, or rectum. In various embodiments, the topical composition is provided in a format selected from a colloid or emulsion (o/w, w/o), cream, lotion, ointment, foam, aqueous or non-aqueous gel, aqueous or non-aqueous solution or suspension, anhydrous suspension, dispersion, paste, or powder. Powder formats include those described above and elsewhere herein, including loose powder, buccal tablet, sublingual tablet, capsule, pouch, lozenge, as well as suppository. As also introduced above, various embodiments of the topical composition may be compounded by combining a powder including the active agent with a base vehicle to compound compositions having a desired format. Additional ingredients, such as those described herein, may also be present in the powder, base vehicle, or mixed therewith.

The base vehicle may be liquid, semi-liquid, or solid. For example, the base vehicle may include an aqueous, anhydrous, organic, or inorganic solution, which may include a dispersion or suspension, cream, gel, ointment, lotion, emulsion, powder, or paste. In some embodiments, the base vehicle includes a base cream, ointment, gel, lotion, foam, or solution. The base vehicle may include base vehicle components such as lecithin, phospholipids, glycols, paraffin, fatty acids, carbopols/carbomers, alcohols, lanolin, for example.

In some embodiments, the base vehicle comprises an aqueous solution. In some examples, base vehicles comprising an aqueous solution may be combined with ingredients of the topical composition to formulate the same. In an embodiment, the base vehicle or component thereof may include an aqueous solution comprising a saline solution. For example, the topical composition may comprise a base vehicle or component comprising a sodium hydroxide solution, which may be a sterile solution, an alcohol, water, e.g., purified water, water for irrigation, water for injection, or a sterile water. In one embodiment, a base vehicle or component thereof comprises a sodium chloride 0.09% solution (sterile). The base vehicle or component may be present in an amount sufficient to obtain the desired amount of active agents per unit weight or volume.

In some embodiments, the topical composition may include a base vehicle comprising a polyethylene glycol (PEG) base vehicle component. In other embodiments, the topical composition is PEG-free. In these or other embodiments, the topical composition may include a silicon or silicon variant base vehicle component. In some embodiments, the topical composition is silicon-free. An example composition may comprise a solution including base vehicle components selected from water, alcohol, DMSO, saline or sodium chloride, sodium hypochlorite, or other aqueous or anhydrous base vehicle medium into which the one or more of the topical composition ingredients are mixed, dispersed, suspended, solubilized, or dissolved. The topical composition may be water soluble/miscible or formulated for water absorption. The topical composition may comprise a water-in-oil emulsion or oil-in-water emulsion. In one embodiment, the topical composition comprises a emulsion, e.g., a cream or lotion format, comprising one or more base vehicle components selected from of acrylate copolymer, alcohol, camphor, carbomer, dimethyl isosorbide, disodium EDTA, dl-alphatocopheryl acetate, edetate disodium, emulsifying wax, eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate (e.g., polysorbate 85, polysorbate 20), purified water, titanium dioxide, tridecyl stearate, tridecyl trimellitate, sodium hydroxide, sodium hydroxide, sorbitol, stearic acid, zinc pyrithione, or combinations thereof. In some embodiments, the topical composition comprises a foam format that includes a propellant base vehicle component such as butane. Compositions comprising a foam format may also comprise additional characteristics such as that of an emulsion, such as an oil-in-water emulsion, or gel.

In one example, the topical composition comprises an ointment format comprising base vehicle components selected from hydrophilic petrolatum, white petrolatum, hydrophilic ointment, white ointment, anhydrous lanolin, hydrous lanolin, PEG ointment, or combinations thereof. In an embodiment, the topical composition comprises a gel format. The gel may be an aqueous or anhydrous gel. The gel may include base vehicle components including thickening agents and/or gelling agents such as carbopol, poloxamer, xanthan gum, methylcellulose, carboxymethyl cellulose, hydroxyethyl cellulose, ethylcellulose, gelatin, magnesium aluminum silicate, polyvinyl alcohol, sodium alginate, or combinations thereof.

The topical composition or base vehicle thereof may include base vehicle components such as one or more solubilizers, stabilizers, buffers, tonicity modifiers, bulking agents, viscosity enhancers/reducers, surfactants, chelating agents, adjuvants, or combinations thereof.

In various embodiments, the topical composition or base vehicle thereof comprises one or more glucose polymers such as a starch, cellulose or cellulose derivatives, polydextrose, or combination thereof. Example starches may include sodium starch glycolate, corn starch, pregelatinized starch, or combination thereof. Example celluloses may include hydroxypropyl cellulose, hypermellose, croscarmellose sodium, ethyl cellulose, microcrystalline cellulose, or combination thereof. Povidone such as povidone K30, copovidone, crospovidone, or combination thereof, may also be present. In some embodiments, glycol and/or a sugar alcohol may be present. Example glycols may include polyethylene glycol, propylene glycol, or combination thereof. Example sugar alcohols may include mannitol. Some embodiments may include oxides such as silicon dioxide, titanium dioxide, ferric oxide, or combination thereof. One embodiment may include any of the above and magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof. In one embodiment, the topical composition does not include one or more of starch, cellulose, polydextrose, sodium starch glycolate, corn starch, pregelatinized starch, hydroxypropyl cellulose, hypermellose, croscarmellose sodium, ethyl cellulose, microcrystalline cellulose, povidone, povidone K30, copovidone, crospovidone, polyethylene glycol, propylene glycol, mannitol, silicon dioxide, titanium dioxide, ferric oxide, magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof.

In some embodiments, the base vehicle comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75.

In various embodiments, the topical composition is anhydrous and comprises the active agent in an anhydrous base vehicle. The topical composition may be formulated for self-emulsification in aqueous environments. For example, the topical composition may include ingredients that impart self-emulsification to the topical composition in aqueous environments. In some embodiments, the aqueous environment is an administration site and the topical composition self-emulsifies in situ. The aqueous environment may be provided by biological fluids. In instance, administration to the mouth may expose the topical composition to saliva, thereby driving self-emulsification. In some embodiments, self-emulsification drives liposome and/or mixed micelle formation for transmucosal or transdermal administration.

In some embodiments, the topical composition comprises medium chain triglycerides NF, glycerin USP, glyceryl distearate NF, stearoyl polyoxyl-32 glycerides NF, ascorbyl palmitate NF, vitamin E acetate USP, and active agent. For example, an anhydrous self-emulsifying base vehicle may comprise medium chain triglycerides NF, glycerin USP, glyceryl distearate NF, stearoyl polyoxyl-32 glycerides NF, ascorbyl palmitate NF, vitamin E acetate USP. A flavorant and/or sweetener, such as those described herein, may be included or added to the topical composition. The topical composition may further include a nonionic water-dispersible surfactant for lipid-based formulations to solubilize and enhance the oral bioavailability of poorly water-soluble APIs. The topical composition may self-emulsify in aqueous media, forming a fine dispersion, which may be referred to as a microemulsion (SMEDDS).

In one example, the topical composition may be formulated in a base vehicle configured to spontaneously form a self-emulsifying liposome, which may include micelle or mixed micelles, for improved drug delivery. For instance, the base vehicle may comprise a self-emulsifying anhydrous liposomal or mixed micelle delivery base vehicle and the topical composition may comprise a self-emulsifying liposome formulation. For example, the topical composition may comprise a self-emulsifying liposome formulation designed to form an emulsion upon contact with aqueous fluids, e.g., saliva in a sublingual administration. The topical composition may be anhydrous and self-emulsify in aqueous environments. The topical composition may be formulated to self-emulsify in situ. For instance, administering the topical composition to the oral cavity of a subject may cause the topical composition to self-emulsify when interacting with saliva. This process may occur without addition of mechanical or thermal energy. In various embodiments, the anhydrous self-emulsifying base vehicle comprises a self-emulsifying liposome base vehicle comprising phosphatidylcholine and lysophosphatidylcholine. For example, the base vehicle may include a synergistic combination of about 0.5% w/w to about 10% w/w of phosphatidyl-choline, preferably about 3% w/w, and about 0.1% w/w to about 1% w/w of lysophosphatidylcholine, preferably about 0.36% w/w.

In some embodiments, the base vehicle comprises an anhydrous lipid based vehicle configured for self-emulsification in an aqueous environment. In one example, the base vehicle may comprise caprylic/capric triglyceride. In this or another example, the base vehicle comprises a glyceryl stearate, such as a monostearate, distearate, or both. In either example or another example, the base vehicle comprises phosphatidylcholine, lysophosphatidylcholine, polycarbophil, glyceryl distearate, and glyceryl monostearate. In any of the above or another example, the base vehicle may include a flavorant, e.g., a flavor and/or sweetener, as described herein, such as monk fruit, natural oil flavor, sucralose, or combination thereof. Formulation of the topical composition may comprise combining powder including the active agent, such as one or more GLP-1 receptor agonists, PDE5 inhibitors, selective serotonin receptor agonists, naltrexone, or combination thereof and the base vehicle. The base vehicle may be preformulated prior to combining or one or more ingredients may be further combined. For instance, one or more flavorants or sweeteners may be added before or after combining powder with other components of the base vehicle.

In various embodiments, the topical composition incorporates polycarbophil. Polycarbophil may enhance the topical composition to achieve both prolonged contact time and effective bitterness masking. The prolonged contact time and effective bitterness masking may position the topical composition as a prime choice for sublingual drug delivery.

Additionally or alternatively, the topical composition may incorporate a specialized technology designed to counteract the bitterness often associated with drugs like semaglutide. For example, the topical composition may employ a unique film-forming adhesion technology that combines glyceryl distearate, glyceryl monostearate, and polycarbophil. The combination works synergistically to create a protective film that minimizes the drug's initial and direct contact with the taste buds, thereby reducing bitterness. Optionally, sweeteners and/or flavors may be added to further diminish any residual bitter taste during ingestion. Example sweeteners and flavors may include those identified herein, such as marshmallow oil flavor, sucralose, Siraitia grosvenorii (Monk) fruit, or combination thereof.

In one embodiment, the topical composition utilizes synergistic properties of glyceryl distearate, glyceryl monostearate, and polycarbophil. For instance, when applied sublingually, these ingredients collaboratively form a protective matrix. This matrix firmly adheres to the sublingual mucosa, thereby prolonging the formulation's contact with the mucosal surface. The extended contact time, primarily facilitated by polycarbophil, enhances the absorption of active agents through the sublingual route, leading to increased bioavailability and optimized therapeutic efficacy. Concurrently, glyceryl distearate and glyceryl monostearate form an anhydrous film that not only improves drug contact time but also minimizes the drug's interaction with taste buds, reducing bitterness. The combination of polycarbophil, glyceryl distearate, and glyceryl monostearate also acts as an effective bitterness-mitigating agent, skillfully masking the bitter taste of certain active agents.

Table 1 provides an example base vehicle for combination with an active agent active agent, such as one or more GLP-1 receptor agonists, PDE5 inhibitors, selective serotonin receptor agonists, naltrexone, or combination thereof, to generate a self-emulsifying suspension. The suspension may be anhydrous and be configured to self-emulsify within an aqueous environment, e.g., in situ, which may be provided by biological fluid at the administration site. The base vehicle in Table 1 is also formulated for taste masking and adhesion as introduced above.

TABLE 1
Anhydrous Suspension Vehicle

| Material Name | INCI | % Material | % Range | Exemplary |
|---|---|---|---|---|
| GELEOL MONO AND | GLYCERYL | 2 | 0.5-5 | 2 |
| DIGLYCERIDES NF | MONOSTEARATE | | | |
| PRECIROL ATO5 | GLYCERYL DISTEARATE | 1 | 0.5-5 | 1 |
| GELUCIRE 48/16 | PEG-32 STEARATE | 3 | 1-5 | 3 |
| LABRAFIL M1944 | APRICOT KERNEL OIL | 1.5 | 0.2-5 | 1.5 |
| | PEG-6 ESTERS | | | |
| PUREFRUIT SELECT | SIRAITIA GROSVENORII | 1.3 | 0.5-2 | 1.3 |
| | FRUIT EXTRACT | | | |
| SUCRALOSE | SUCRALOSE | 1 | 0.0-2 | 1 |
| CAPRYLIC/CAPRIC | CAPRYLIC/CAPRIC | 80.6 | 60-95 | 80.4 |
| TRIGLYCERIDES | TRIGLYCERIDE | | | |
| PHOSAL 53 MCT | LECITHIN | 6 | 1-10 | 6 |
| | CAPRYLIC/CAPRIC | | | |
| | TRIGLYCERIDE | | | |
| | ALCOHOL | | | |
| | GLYCERYL STEARATE | | | |
| | OLEIC ACID | | | |
| | ASCORBYL PALMITATE | | | |
| | TOCOPHEROL | | | |
| NOVEON AA-1 | POLYCARBOPHIL USP | 0.6 | 0.2-2 | 0.6 |
| POLYCARBOPHIL | | | | |
| MARSHMALLOW O.S NAT | NATURAL FLAVOR | 3 | 1-5 | 3 |
| | | (SUM) 100 | | |

The ingredient PHOSAL 53 MCT contains a minimum of 53% of phosphatidylcholine and a maximum of 6% lysophosphatidylcholine. In some embodiments, the base vehicle includes a synergistic combination of about 0.5% w/w to about 10% w/w of phosphatidylcholine and about 0.1% w/w to about 1% w/w of lysophosphatidylcholine. A current preferred formulation includes about 3% w/w phosphatidylcholine and about 0.36% w/w lysophosphatidylcholine. As described above and elsewhere herein flavorant may be optional and may be added separately. One or more ingredients may also be excluded or replaced by pharmaceutical ingredients determined to be equivalent. In the formulations exemplified below (Tables 2-10), the self-emulsifying liposome suspension vehicle consistent with Table 1 is utilized without flavorant, which is added separately in certain of the formulations.

Table 2 illustrates an example compounding procedure to formulate a topical composition comprising a semaglutide suspension. As shown, the base vehicle comprises an anhydrous self-emulsifying liposome suspension vehicle (Table 1) which may also be referred to as a preliposome vehicle; however, other base vehicles described herein may also be used. The base vehicle may be lipid based and form a suspension when mixed with an insoluble active agent. When administered to an aqueous environment or otherwise mixed with aqueous fluid, the suspension self-emulsifies to form a mixed micelle liposomal delivery system. The procedure includes mixing ground commercial oral semaglutide tablets (RYBELSUS® 14 mg) and base vehicle (Table 1). The tablets were ground separately. Ingredients were weighted using an appropriate balance. The ingredients where combined in an electronic mortar and pestle container and mixed once on normal in an Ungator 2100. The viscosity preferably ranges from 100 to 1500 CPS and while depend on inclusion or exclusion of wetting agents and oil flavors.

TABLE 2
Semaglutide Suspension Formula

| | |
|---|---|
| Semaglutide tablet, ground | Q.S. |
| Base Self-emulsifying liposome | Q.S. |
| suspension | |
| Wetting agent (optional) | 2 to 10%, preferred 5% |
| Flavor (optional) | 0.2-3%, preferred 1% |

Table 3 illustrates an example compounding procedure to formulate a topical composition comprising a 2 mg/mL semaglutide suspension. A RYBELSUS® (Semaglutide) 14 mg tablet is ground to a powder. Each mL of the suspension contains 2 mg of semaglutide equivalent to 2/14 or 14.29% of each tablet. With an average tablet weight is 409 mg, each mL of suspension includes 58.429 mg of semaglutide tablet, equivalent to 2 mg semaglutide. Thus, for each mL of suspension, add 58.429 mg of powder, mix with base vehicle, which is an anhydrous self-emulsifying liposome suspension vehicle (Table 1) in this example, then q.s. to total volume, which may include about 0.94 gm of the base vehicle per mL. The consistency of the suspension was homogenous. The composition may be administered in suitable volumes such as 0.5 mL, 1.0 mL, 1.5 mL, 2.0 mL, or as otherwise prescribed. The composition may preferably be retained in the mouth for at least 5 minutes prior to swallowing. Preparation of tablet powder and mixing may be performed in a manner similar to that described with respect to Table 2.

TABLE 3
Semaglutide 2 mg/mL Suspension Formula

| | |
|---|---|
| Semaglutide tablet, ground | Powder equivalent to 2 mg |
| | Semaglutide per mL |
| Base Self-emulsifying liposome | Q.S. |
| suspension vehicle | |

As described above, the topical composition may include flavoring. Table 4 illustrates an example compounding procedure to formulate a topical composition comprising a 2 mg/ml semaglutide suspension including flavoring. A RYBELSUS® (Semaglutide) 14 mg tablet is ground to a powder. Each mL of the suspension contains 2 mg of semaglutide equivalent to 2/14 or 14.29% of each tablet. With an average tablet weight is 409 mg, each mL of suspension includes 58.429 mg of powder, equivalent to 2 mg semaglutide. Thus, for each mL of suspension, add 58.429 mg of semaglutide tablet, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.94 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. The composition may be administered in suitable volumes such as 0.5 mL, 1.0 mL, 1.5 mL, 2.0 mL, or as otherwise prescribed. The composition may preferably be retained in the mouth for at least 5 minutes prior to swallowing.

TABLE 4
Semaglutide 2 mg/mL Flavored Suspension Formula

| | |
|---|---|
| Semaglutide tablet, ground | Powder equivalent to 2 mg Semaglutide |
| Flavor, Peppermint Oil | 0.0009 mL |
| Flavor, Spearmint Oil | 0.0009 mL |
| Base Self-emulsifying liposome | Q.S. |
| suspension vehicle | |

Table 5 illustrates an example compounding procedure to formulate a topical composition comprising a 2.5 mg/mL tadalafil and 2 mg/mL semaglutide suspension including flavoring. A RYBELSUS® (Semaglutide) 14 mg tablet is ground to a powder. Each mL of the suspension contains 2 mg of semaglutide equivalent to 2/14 or 14.29% of each tablet. With an average tablet weight is 409 mg, each mL of suspension includes 58.429 mg of powder, equivalent to 2 mg semaglutide. A Tadalafil 20 mg tablet is ground to powder. Each mL of the suspension contains 2.5 mg of tadalafil equivalent to 2.5/20 or 12.5% of each tablet. With an average weight of 368 mg, each mL of suspension include 46 mg of powder, equivalent to 2.5 mg tadalafil. Thus, for each mL of suspension, add 58.429 mg of semaglutide tablet powder and 46 mg of tadalafil tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.89 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for at least 5 minutes prior to swallowing.

TABLE 5
Tadalafil 2.5 mg/mL - Semaglutide 2 mg/mL Flavored
Suspension Formula

| | |
|---|---|
| Semaglutide tablet, ground | Powder equivalent to 2 mg Semaglutide |
| | per mL |
| Tadalafil tablet, ground | Powder equivalent to 2.5 mg Tadalafil per mL |
| Flavor, Peppermint Oil | 0.0009 mL |
| Flavor, Spearmint Oil | 0.0009 mL |
| Base Self-emulsifying | Q.S. |
| liposome | |
| suspension vehicle | |

Table 6 illustrates an example compounding procedure to formulate a topical composition comprising a 2.5 mg/mL tadalafil. A Tadalafil 20 mg tablet is ground to powder. Each mL of the suspension contains 2.5 mg of tadalafil equivalent to 2.5/20 or 12.5% of each tablet. With an average weight of 360 mg, each mL of suspension include 45 mg of powder, equivalent to 2.5 mg tadalafil. Thus, for each mL of suspension, add 45 mg of tadalafil tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.95 gm of the base vehicle per mL. The consistency of the suspension was homogenous. In another example, a composition include 5 mg/mL tadalafil may include 90 mg of tadalafil tablet powder per mL and about 0.908 gm base vehicle. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for at least 2 minutes prior to swallowing.

TABLE 6
Tadalafil 2.5 mg/mL Flavored Suspension Formula

| | |
|---|---|
| Tadalafil tablet, ground | Powder equivalent to 2.5 mg Tadalafil per mL |
| Flavor, Peppermint Oil | 0.0009 mL |
| Flavor, Spearmint Oil | 0.0009 mL |
| Base Self-emulsifying | Q.S. |
| liposome | |
| suspension vehicle | |

Table 7 illustrates an example compounding procedure to formulate a topical composition comprising a 10 mg/mL vardenafil. A Vardenafil 20 mg tablet is ground to powder. Each mL of the suspension contains 10 mg of vardenafil equivalent to 10/20 or 50% of each tablet. With an average weight of 276 mg, each mL of suspension includes 138 mg of vardenafil tablet powder, equivalent to 10 mg vardenafil. Thus, for each mL of suspension, add 138 mg of vardenafil tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.86 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for at least 5 minutes prior to swallowing.

TABLE 7
Vardenafil 10 mg/mL Flavored Suspension Formula

| | |
|---|---|
| Vardenafil tablet, ground | Powder equivalent to 10 mg Vardenafil per mL |
| Flavor, Peppermint Oil | 0.0009 mL |
| Flavor, Spearmint Oil | 0.0009 mL |
| Base Self-emulsifying | Q.S. |
| liposome | |
| suspension vehicle | |

Table 8 illustrates an example compounding procedure to formulate a topical composition comprising a 25 mg/mL sumatriptan. A Sumatriptan 100 mg tablet is ground to powder. Each mL of the suspension contains 25 mg of sumatriptan equivalent to 25/100 or 25% of each tablet. With an average weight of 410 mg, each mL of suspension includes 103 mg of sumatriptan tablet powder, equivalent to 25 mg sumatriptan. Thus, for each mL of suspension, add 103 mg of sumatriptan tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.896 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed, e.g., for headaches. The composition may preferably be retained in the mouth for at least 2 minutes prior to swallowing.

TABLE 8
Sumatriptan 25 mg/mL Flavored Suspension Formula

| | |
|---|---|
| Sumatriptan tablet, ground | Powder equivalent to 25 mg |
| | Sumatriptan per mL |
| Flavor, Peppermint Oil | 0.0009 mL |
| Flavor, Spearmint Oil | 0.0009 mL |
| Base Self-emulsifying liposome | Q.S. |
| suspension vehicle | |

Table 9 illustrates an example compounding procedure to formulate a topical composition comprising a 6 mg/mL naltrexone. A Naltrexone HCl 50 mg tablet is ground to powder. Each mL of the suspension contains 6 mg of naltrexone HCl equivalent to 6/50 or 12% of each tablet. With an average weight of 306 mg, each mL of suspension includes 36.72 mg of naltrexone HCl tablet powder, equivalent to 6 mg naltrexone HCl. Thus, for each mL of suspension, add 36.72 mg of naltrexone HCl tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.96 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for 2 minutes prior to swallowing.

TABLE 9
Naltrexone 6 mg/mL Flavored Suspension Formula

| | |
|---|---|
| Naltrexone tablet, ground | Powder equivalent to 6 mg |
| | Naltrexone per mL |
| Flavor, Peppermint Oil | 0.0009 mL |
| Flavor, Spearmint Oil | 0.0009 mL |
| Base Self-emulsifying liposome | Q.S. |
| suspension vehicle | |

Table 10 illustrates an example compounding procedure to formulate a topical composition comprising a 9 mg/mL naltrexone. A Naltrexone HCl 50 mg tablet is ground to powder. Each mL of the suspension contains 9 mg of naltrexone HCl equivalent to 9/50 or 18% of each tablet. With an average weight of 306 mg, each mL of suspension includes 55.08 mg of naltrexone HCl tablet powder, equivalent to 9 mg naltrexone HCl. Thus, for each mL of suspension, add 55.08 mg of naltrexone HCl tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.94 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for at least 2 minutes prior to swallowing.

TABLE 10
Naltrexone 9 mg/mL Flavored Suspension Formula

| | |
|---|---|
| Naltrexone tablet, ground | Powder equivalent to 9 mg Naltrexone per mL |
| Flavor, Peppermint Oil | 0.0009 mL |
| Flavor, Spearmint Oil | 0.0009 mL |
| Base Self-emulsifying | Q.S. |
| liposome | |
| suspension vehicle | |

Table 11 illustrates an example compounding procedure to formulate a topical composition comprising a 1 mg/mL semaglutide suspension including flavoring. A RYBELSUS® (Semaglutide) 14 mg tablet is ground to a powder. Each mL of the suspension contains 1 mg of semaglutide equivalent to 1/14 or 7.14% of each tablet. With an average tablet weight is 409 mg, each mL of suspension includes 29.2 mg of powder, equivalent to 1 mg semaglutide. Thus, for each mL of suspension, add 29.2 mg of semaglutide tablet, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.97 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. The composition may be administered in suitable volumes such as 0.5 mL, 1.0 mL, 1.5 mL, 2.0 mL, or as otherwise prescribed. The composition may preferably be retained in the mouth for 5 minutes prior to swallowing.

TABLE 11
Semaglutide 2 mg/mL Flavored Suspension Formula

| | |
|---|---|
| Semaglutide tablet, ground | Powder equivalent to 1 mg Semaglutide |
| Flavor, Peppermint Oil | 0.0009 mL |
| Flavor, Spearmint Oil | 0.0009 mL |
| Base Self-emulsifying liposome | Q.S. |
| suspension vehicle | |

Table 12 illustrates an example compounding procedure to formulate a topical composition comprising a 3 mg/mL semaglutide suspension including flavoring. A RYBELSUS® (Semaglutide) 14 mg tablet is ground to a powder. Each mL of the suspension contains 3 mg of semaglutide equivalent to 3/14 or 21.3% of each tablet. With an average tablet weight is 409 mg, each mL of suspension includes 29.2 mg of powder, equivalent to 1 mg semaglutide. Thus, for each mL of suspension, add 87.6 mg of semaglutide tablet, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.91 gm of the base vehicle per mL. The consistency of the suspension was homogenous. In a further example, the composition includes about 9.6 mg of xylitol and poloxamers, in which case the amount of base vehicle would be similarly reduced. Preparation of tablet powder and mixing may be performed in a manner similar to that described with respect to Table 2. Those skilled in the art will appreciate that other flavoring, flavoring combinations, and flavoring amounts may be used. The composition may be administered in suitable volumes such as 0.5 mL, 1.0 mL, 1.5 mL, 2.0 mL, or as otherwise prescribed. The composition may preferably be retained in the mouth for 5 minutes prior to swallowing.

TABLE 12
Semaglutide 3 mg/mL Flavored Suspension Formula

| | |
|---|---|
| Semaglutide tablet, ground | Powder equivalent to 3 mg Semaglutide |
| Flavor, Peppermint Oil | 0.0009 mL |
| Flavor, Spearmint Oil | 0.0009 mL |
| Base Self-emulsifying liposome | Q.S. |
| suspension vehicle | |

Table 13 illustrates an example compounding procedure to formulate a topical composition comprising a 2 mg/mL naltrexone. A Naltrexone HCl 50 mg tablet is ground to powder. Each mL of the suspension contains 2 mg of naltrexone HCl equivalent to 1/25 or 4% of each tablet. With an average weight of 306 mg, each mL of suspension includes 13.2 mg of naltrexone HCl tablet powder, equivalent to 2 mg naltrexone HCl. Thus, for each mL of suspension, add 13.2 mg of naltrexone HCl tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.987 gm of the base vehicle per mL. The consistency of the suspension was homogenous. In another example, the strength is 3 mg/mL, and each mL includes 18.36 mg of tablet powder and about 0.98 gm of the base vehicle. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for 2 minutes prior to swallowing.

TABLE 13
Naltrexone 2 mg/mL Flavored Suspension Formula

| | |
|---|---|
| Naltrexone tablet, ground | Powder equivalent to 6 mg Naltrexone per mL |
| Flavor, Peppermint Oil | 0.0009 mL |
| Flavor, Spearmint Oil | 0.0009 mL |
| Base Self-emulsifying | Q.S. |
| liposome | |
| suspension vehicle | |

Table 14 illustrates an example compounding procedure to formulate a topical composition comprising a 15 mg/mL sildenafil. A Sildenafil 100 mg tablet is ground to powder. Each mL of the suspension contains 15 mg of sildenafil equivalent to 15% of each tablet. With an average weight of 641 mg, each mL of suspension includes 96.2 mg of sildenafil tablet powder, equivalent to 15 mg sildenafil. Thus, for each mL of suspension, add 96.2 mg of sildenafil tablet powder, mix with 0.0009 mL of peppermint oil NF, 0.0009 mL of spearmint oil flavor, and base vehicle, which is an anhydrous self-emulsifying liposome suspension vehicle (Table 1) in this example, then q.s. with the base vehicle to total volume, which may include about 0.9 gm of the base vehicle per mL. The consistency of the suspension was homogenous. Preparation of tablet powder and mixing may be performed in a manner similar to that described with respect to Table 2. Those skilled in the art will appreciate that other flavoring, flavoring combinations, and flavoring amounts may be used. Flavoring may also be optional. The composition may be administered in suitable volumes such as 0.5 mL, 1.0 mL, 1.5 mL, 2.0 mL, or as otherwise prescribed. The composition may preferably be retained in the mouth for at least 2 minutes or at least 5 minutes prior to swallowing.

TABLE 14
Sildenafil 15 mg/mL Flavored Suspension Formula

| | |
|---|---|
| Sildenafil tablet, ground | Powder equivalent to 15 mg |
| | Sildenafil per mL |
| Flavor, Peppermint Oil | 0.0009 mL |
| Flavor, Spearmint Oil | 0.0009 mL |
| Base Self-emulsifying liposome | Q.S. |
| suspension vehicle | |

Those having skill in the art will appreciate that the compositions described herein are not limited to compounding utilizing ground tablets. For example, a composition formulated from bulk powder may be prepared and delivered in a manner similar to that described above. In one example, noting that sildenafil 1 mg is equivalent to Sildenafil Citrate USP 1.4 mg, a sildenafil 50 mg/ml composition includes, per mL, 70 mg sildenafil citrate powder and about 1 mL base vehicle, such as the base vehicle or similar described with respect to Table 1 or elsewhere herein. The composition may include flavoring. For instance, the composition may include 0.001 mL peppermint oil, 0.001 mL spearmint oil flavor, or both. Similarly, a composition comprising 10 mg/mL tadalafil may be formulated with 10 mg tadalafil powder and about 1 mL base vehicle, such as the base vehicle or similar described with respect to Table 1 or elsewhere herein. The composition may include flavoring. For instance, the composition may include 0.001 mL peppermint oil, 0.001 mL spearmint oil flavor, or both.

A variety of other compositions may similarly be formulated with other active ingredients. For instance, a composition for oral absorption to treat nausea or vomiting may include ondansetron and the base vehicle. The composition may be formulated in various strengths such as between 2 mg/mL and 20 mg/mL. For example, an ondansetron 8 mg/mL composition may be formulated with powder of an Ondansetron 8 mg ODT tablet. The tablet has an average weight of 100 mg. This powder may be mixed with an appropriate amount of base vehicle. For example, each mL may include about 100 mg of Ondansetron 8 mg ODT tablet powder and about 0.9 gm base vehicle per mL. The composition may include flavoring. For instance, the composition may include 0.001 mL peppermint oil, 0.001 mL spearmint oil flavor, or both.

A method of making the topical composition may comprise mixing powder including the active agent, such as one or more GLP-1 receptor agonists, PDE5 inhibitors, selective serotonin receptor agonists, naltrexone, or combination thereof, with a base vehicle. The base vehicle may be anhydrous in a liquid state including lipids. The base vehicle may be an anhydrous base vehicle in a liquid state including phosphatidylcholine and lysophosphatidylcholine. The base vehicle may include triglycerides. The triglycerides may include medium chain triglycerides such as caprylic/capric triglycerides. The base vehicle may additionally or alternatively include polycarbophil. The base vehicle may additionally or alternatively include glyceryl distearate and glyceryl monostearate. The base vehicle may be self-emulsifying as described herein. The base vehicle may be a self-emulsifying liposome suspension vehicle that is anhydrous and includes phosphatidylcholine and lysophosphatidylcholine, e.g., about 0.5% w/w to about 10% w/w of phosphatidylcholine and about 0.1% w/w to about 1% w/w of lysophosphatidylcholine, such as about 3% w/w phosphatidylcholine and about 0.36% w/w lysophosphatidylcholine. This self-emulsifying liposome suspension vehicle may be used in the formulations described above with respect to Tables 2-10 or any other formulation described herein. In some embodiments, the self-emulsifying liposome suspension vehicle may further include polycarbophil, glyceryl distearate, and glyceryl monostearate, or suitable equivalents thereof.

The self-emulsifying liposome suspension vehicle may comprise a base vehicle as described in Table 1 or elsewhere herein. To prepare the topical composition, the user may triturate the powder. The powder may optionally be wetted with a wetting agent. Example wetting agents include propylene glycol or glycerin. Upon adding the base vehicle and mixing, the topical composition's solubility in the lipid suspension is enhanced. When applied sublingually, it spontaneously forms a self-emulsifying liposome suspension, improving drug delivery. For even more refined preliposome formation, an electric mortar and pestle may be utilized after addition of active agent. This tool can offer a more consistent and thorough mixing, ensuring the highest quality of the end product.

In various embodiments, the topical composition may be administered topically by contacting an external surface of the body, which may include skin or a vaginal or anal orifice, for absorption there along. The topical composition may be administered to the oral cavity or nasal cavity for absorption therein. The topical composition may be administered in a spray, coat, soak, powder, spread, or the like, for example, suitable to the topical format.

A method of compounding the topical composition may include obtaining all or a portion of the active agent, such as one or more GLP-1 receptor agonists, PDE5 inhibitors, selective serotonin receptor agonists, naltrexone, or combination thereof, alone or together with one or more additional ingredients from a powder format, e.g., from bulk powder, ground tablet, injection powder, or other commercially available composition. When tablet powder is used, the amount of ground powder will typically be equivalent to the weight of the tablet multiplied by the desired amount of active in the formulation or unit thereof divided by in the amount of active in the ground powder. For example, if 3 tablets, each containing 10 mg of active, are ground to a powder, and each tablet weights 40 mg or the total powder otherwise weighs 120 mg, 1 mg desired active agent divided by 30 mg active agent, is about 0.33. Multiplied by the total weight of the powder, the powder contains 1 mg of active in every 4 mg. The powder may be mixed with a base vehicle, such as those described herein. The method may include combining the base vehicle and a powder containing all or a portion of the active agent. For example, the active agent may be obtained from commercial tablets, ground to a desired particle size. In one embodiment, all or a portion of the active agent may be obtained from a commercially manufactured injection powder or solution and mixed with the base vehicle to compound the topical composition. In one example, active agent is wetted prior to combining with the base vehicle. For instance, according to various compounding methods, the powder may be wetted with DMSO, alcohol, or water. In one embodiment, the powder may be wetted with propylene glycol or glycerin. According to a method, all or a portion of additional ingredients may be provided in a format selected from a solution, emulsion, gel, cream, lotion, ointment, or other format and may be combined with the base vehicle together with or separate of all or a portion of the active agent. In one example, all or a portion of additional ingredients may be mixed with all or a portion of the active agent prior to being mixed with other ingredients of the base vehicle. In another example, the active agent is added to additional ingredients that are provided in a commercially available medicated composition comprising the base vehicle. The base vehicle may comprise a commercially available base vehicle including one or more additional ingredients and a powder or other format including the active agent may be mixed therewith to compound the topical composition. For instance, a suspending base vehicle including a flavorant may be mixed with active agent for administration to the oral cavity via oral absorption. In one example, the active agent is obtained from ground commercial oral tablets for oral administration. The powder may be mixed with a base vehicle comprising phospholipids and/or preliposomes for subsequent reconstitution with liquid base vehicle components. The powder may be mixed with a phospholipid base vehicle to formulate a liposomal or mixed micelle system. In one example, the powder is mixed with a suspension base vehicle including a liposomal or mixed micelle system. In one formulation the base vehicle is an anhydrous liquid. The base vehicle may be anhydrous in a liquid state including lipids. The base vehicle may be an anhydrous base vehicle in a liquid state including phosphatidylcholine and lysophosphatidylcholine. The base vehicle may include triglycerides, such as medium chain triglycerides. The triglycerides may comprises or consist of caprylic/capric triglycerides. The base vehicle may be self-emulsifying as described herein. The base vehicle may additionally include one or more of polycarbophil, glyceryl distearate, and glyceryl monostearate. In a further example, the base vehicle optionally includes a flavorant or a flavorant is added. The base vehicle may comprise a base vehicle as described in Table 1 or elsewhere herein.

As described above and elsewhere herein, the base vehicle may comprise a base vehicle configured for self-emulsification in an aqueous environment. Such a base vehicle may be an anhydrous self-emulsifying suspension vehicle. The anhydrous self-emulsifying base vehicle may comprise an anhydrous lipid-based self-emulsifying suspension vehicle. The base vehicle may form a suspension upon mixing with an active or other agent insoluble therein. When administered to an aqueous environment or mixed with an aqueous liquid, the suspension may self-emulsify to form a suspension comprising a mixed micelles liposome system. Active agent may be encapsulated by lipid aggregates, e.g., micelles and liposomes, or associated with membranes thereof. Hydrophobic active agents such as semaglutide, tadalafil, and vardenafil may not be inherently soluble in the anhydrous base vehicle. Self-emulsification may drive enhanced solubilization. Hydrophilic active agents such as naltrexone HCl and sumatriptan may not be soluble in the anhydrous base. Utilizing the base vehicle described herein, the base provides a unique role as a preliposome system designed to form delivery structures in situ. The base vehicle may employ a preliposome concept. For example, in such an anhydrous preliposome suspension vehicle, liposome formation occurs upon self-emulsification, providing a mechanism for encapsulating and enhancing the bioavailability of active agents. With respect to loading and association, solubilization of hydrophobic agents may be driven by association with lipid bilayers during emulsification. Improved permeability provided by the base vehicle may be driven by facilitating this bilayer integration. Encapsulation of hydrophilic agents within liposome cores during emulsification may ensures dispersion and protection, enhancing absorption and bioavailability. The base vehicle includes phosphatidylcholine 0.1-10% w/w and lysophosphatidylcholine 0.01-1% w/w. These concentrations drive micelle and liposome formation. The topical composition may include the base vehicle in an amount 85% or greater to ensure adequate precursor material for the in situ formation of liposomes and the introduction of active agents. In one example, the topical composition includes phosphatidylcholine 0.85-10% w/w and lysophosphatidylcholine 0.0085-1% w/w. In one example, the topical composition includes phosphatidylcholine 0.425-10% w/w and lysophosphatidylcholine 0.085-1% w/w. The topical composition may comprise 50-95% triglycerides. The triglycerides may comprise or consist of medium chain triglycerides. The medium chain triglycerides may comprise or consist of caprylic/capric triglycerides. The topical composition may additionally include glyceryl distearate 0.425-5% w/w and glyceryl monostearate 0.425-5% w/w. The topical composition may comprise polycarbophil 0.17-2%. The base vehicle of the topical composition may also be referred to as a preliposome micelle hybrid vehicle, which may accurately describe an intermediate state of the suspension before full liposome formation. Solubilization of active agents may rely, at least in part, upon the presence of surfactants and their function in the formation of micelles. The incorporation of cholesterol described herein may be utilized to further improve the flexibility of micelles, which facilitates the solubilization and dispersion of hydrophobic agents such as semaglutide, tadalafil, and vardenafil, as well as hydrophilic agents like naltrexone HCl and sumatriptan. The topical composition may additionally include microemulsion characteristics. For instance, the base vehicle may be referred to as a self-microemulsifying drug delivery system (SMEDDS) hybrid that improves drug solubilization, stability, and permeability by the self emulsion characteristics resulting in superior drug delivery outcomes. By utilizing a self-microemulsifying drug delivery system to maximize drug delivery according to the present disclosure, the technology demonstrates versatility in managing both hydrophobic and hydrophilic active agents.

As noted above, the base vehicle may incorporate cholesterol for improved functionalities. Cholesterol may be present in the base vehicle in an amount between 0.01% and 0.5% w/w. Cholesterol may be present in the topical composition in an amount between 0.0085% and 0.5% w/w. For example, malleability of micelles and liposomes may be substantially improved by the inclusion of cholesterol in the formulation, which may provide functionalities comparable to transferosome technology. In lipid bilayers, for example, cholesterol may function as a stabilizing agent, thereby reducing rigidity and facilitating the development of more adaptable, deformable structures. This may be particularly beneficial for micelles wherein capacity to traverse biological barriers, such as mucosal membranes, is enhanced by an increase in flexibility. The delivery and bioavailability of active agents are also improved by the enhanced deformability of liposomes, which enables greater interaction with and penetration through the oral mucosa. The presence of cholesterol also contributes to the stability of micelles, thereby reducing the probability of premature collapse or aggregation prior to emulsification. Thus, cholesterol may be included to improve permeability and absorption, as it enhances the adaptability and structural integrity of the delivery system. As described above and elsewhere herein, the base vehicle may additionally or alternatively include polycarbophil, glyceryl distearate, and glyceryl monostearate configured to form a protective matrix/film that adheres to the mucosa to prolong contact while also minimizing initial drug contact with tastebuds to reduce bitterness.

The topical composition may include an anhydrous liquid format that leverages amphiphilic components (lipids, surfactants, and stabilizers, such as cholesterol and phosphatidylcholine) to create a unique in-situ self-assembling delivery mechanism when the anhydrous suspension is contacted with water. The system remains anhydrous until contacted with water, where it spontaneously forms mixed micelles and/or liposomes. The topical composition is designed to offer significant advantages in solubilization, stability, and targeted delivery for pharmaceuticals, nutraceuticals, and other applications. For example the topical composition may include amphiphilic components in liquid form, which may be provided by the suspension base vehicle described herein, and include lipids, surfactants, and stabilizers. The lipid components are configured to provide a structural basis for liposome formation. Surfactant components are configured to facilitate micelle formation and stabilize the system. The stabilizer components, e.g., cholesterol and phosphatidylcholine, are configured to enhance membrane integrity and self-assembly dynamics. Upon contact with water (e.g., saliva or gastric fluid), the amphiphilic molecules organize spontaneously into mixed micelles comprising monolayer structures that solubilize hydrophobic compounds for rapid absorption. Contact with water further drives spontaneous formation of liposomes. These bilayer vesicles encapsulate hydrophilic compounds for controlled or targeted release. Thus, upon hydration, the liquid system of the topical composition produces a dual acting system. For example, upon hydration (e.g., by saliva), the liquid system rapidly forms mixed micelles and liposomes to facilitate efficient drug solubilization and absorption. The mixed micelles enhance the solubilization of lipophilic compounds, while the liposomes contribute to stabilizing the formulation.

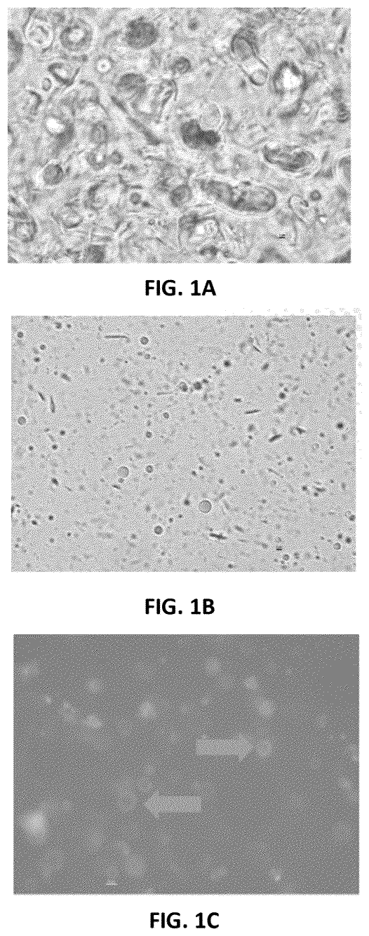

FIG. 1A is a image of a topical composition according to various under magnification. The topical composition comprises a base vehicle and semaglutide 1 mg/ml anhydrous liquid suspension. The base vehicle is an anhydrous base vehicle consistent with Table 1, without flavorant. FIG. 1B shows the suspension of FIG. 1A following contact with water. Addition of water drives self-emulsification that forms a mixed micelle liposome system.

To visualize the topical composition following addition of water for self-emulsification, green fluorescent protein (GFP) to represent the peptide semaglutide, was added to the base vehicle comprising an anhydrous base vehicle consistent with Table 1, without flavorant. A GFP 0.1 mg/mL suspension was formulated and diluted 1:1 with water with gentle mixing to mimic administration and contact of the formulation with saliva. The distribution of GFP in the diluted suspension was observed under microscopy using blue light at 40× magnification. FIG. 1C shows a florescent microscopy image of the resulting dilution of the suspension. Without being bound to theory, exposure to water drives spontaneous formation of vesicles (mixed micelles and liposomes). The GFP is encapsulated inside cores and distributed on membranes of the vesicles. The spontaneous micellar and liposome formation when contacted with water is a favorable attribute for the delivery of active agents. The formation avoids instability issues often associated with micelles and liposomes. The liposomes contain lipid bilayers composed of phospholipids and cholesterols, mimicking the structure of cell membranes. Thus, micelles and liposomes can fuse with cell membranes to release the active agent instead of relying on endocytosis. This mechanism may be used to ensure rapid drug delivery, independent of drug molecular size, and reduces risk of drug degradation.

In some embodiments, the topical composition comprising an anhydrous self-emulsifying composition may be combined with aqueous liquid prior to administration to drive emulsification. In one embodiment, the emulsification may generate a microemulsion. In one embodiment, the emulsification generates a liposomal or mixed micelle system.

The number of tablets used to prepare the active agent for compounding to compound the topical composition comprising a desired weight of active agent may be determined by dividing the weight of active agent needed by the weight of active agent in a tablet. The weight of tablet powder needed to formulate a composition comprising a desired weight of the active agent may be determined by dividing the weight of a tablet by the weight of the active agent present in the tablet and multiplying the result by the desired weight of active agent in the topical composition.

In one embodiment, a method of compounding the topical composition includes crushing or obtaining powder from ground oral commercial tablets comprising the active agent. In one example, the topical composition is formulated from commercial oral semaglutide tablets. Tablets may be ground, preferably separately from the vehicle. Any desired strength formulations may be formulated according to the present description. For instance, an oral tablet may be weighed to determine the amount of semaglutide per measurement unit. This formula may then be used to determine the amount of tablet powder needed for combination with vehicle and any additional ingredients to obtain the desired percent composition. For example, using semaglutide as an example active agent, a 14 mg RYBELSUS® tablet has an average weight of 0.409 gm. To formulate a semaglutide 1 mg/ml composition using powder from the 14 mg semaglutide oral tablet, each ml of the topical composition includes 1 mg of semaglutide, which is 1/14 or 7.14% of one tablet. Thus, each mL of the topical composition includes 29.214 mg of tablet powder and the desired concentration may be obtained by mixing the powder with vehicle and any other ingredients to the volume corresponding to the weight of tablet powder for the desired concentration. The appropriate amount of powder including the desired amount of active agent may be mixed with the desired base vehicle. The base vehicle may be any base vehicle described herein. For example, the base vehicle may be an aqueous, non-aqueous, anhydrous, emulsion, liquid, semi-solid, or solid base vehicle. Mixing the base vehicle and ground active agent tablet powder may formulate a power, solution, suspension, ointment, cream, gel, or paste. In one embodiment, a resulting powder may be compressed into a tablet format or encased in a capsule or pouch for administration to the mouth via buccal, sublingual, or other oral absorption delivery. In one embodiment, the mixture may be fully or partially solidified to generate a gel or oral absorption lozenge. In one embodiment, the base vehicle comprises an anhydrous suspension base vehicle. In a further embodiment, the base vehicle comprises an anhydrous suspension base vehicle including liposomes. As described herein the liposomes may be within a mixed micelle system or may be provided pure such that the system includes aggregate lipids that are mainly or substantially associated in bilayers. In one embodiment, the vehicle comprises a mixed micelle system free of liposomes. In one example formulation the suspension base vehicle is formulated to self-emulsify in an aqueous environment. In one embodiment, the base vehicle comprises an anhydrous suspension base vehicle and is mixed with a powder comprising the ground active agent tablets and preliposomes. In one embodiment, the anhydrous suspension or powder includes mixed micelles, with or without preliposomes. The base vehicle, powders, or mixture may include or be combined with additional ingredients, such as those described herein. In one example, the additional ingredients include a flavorant and/or sweetener. For example, the flavorant may include a sweetener to mask the bitter taste of semaglutide, other GLP-1 receptor agonist, or other active agent. In one embodiment, the flavorant and/or sweetener may be added to the ground tablet powder. For example, compounding the topical composition may include triturating the flavorant, e.g., sweetener powder, with the ground tablet powder and then mix with the base vehicle, which may be an anhydrous. In one embodiment, the flavorant may be embedded in the base vehicle. For example, the flavorant, e.g., sweetener powder, may be embedded in the base vehicle and compounding the topical composition includes combining the ground tablet powder with the base vehicle. In some embodiments, the flavorant may be provided as a liquid. In one example, the flavorant may be used to wet the ground tablet powder.

The present disclosure describes various embodiments of compositions including active agents, such as one or more GLP-1 receptor agonists, PDE5 inhibitors, selective serotonin receptor agonists, naltrexone, or combination thereof. However, it is to be understood that such embodiments may additionally or alternatively include other active agents. Thus, all the various embodiments including particular active agents are additionally or alternatively disclosed as including one or more additional active agents. The active agents may include independently or in combination one or more of semaglutide, liraglutide, exenatide, lixisenatide, taspoglutide, lotiglipron, dulaglutide, tirzepatide, albiglutide, danuglipron, orforglipron, efpeglenatide, vardenafil, tadalafil, sildenafil, avanafil, almotriptan, eletriptan, frovatriptan, naratriptan, rizatriptan, sumatriptan, zolmitriptan, naltrexone, or other active agents. The chemical compounds, active agents, and other ingredients described herein are to be understood as encompassing derivatives, analogs, and pharmaceutically acceptable equivalents unless indicated otherwise. Base vehicles described herein may refer to the excipient portion of a topical composition and does not necessarily refer to a set of ingredients that are combined prior to addition of an active agent. Indeed, ingredients of a base vehicle may be combined with the base vehicle (other ingredients) in whatever order is suitable for formulation of the topical composition. For example, when included, flavorants and/or sweeteners may be combined with active agents, other base vehicle ingredients, or combinations thereof independently or in combinations in any order that achieves the flavorant and/or sweetener function together with the functions of the topical composition.

Embodiments described herein that include self-emulsifying base vehicles or that are otherwise formulated to self-emulsify in an aqueous environments may be topically administered as described herein. For example, the topical composition may be administered for transdermal or transmucosal delivery. The topical composition may be administered to the oral cavity, anus, rectum, nasal cavity, ear, or vagina.

The topical composition comprising the anhydrous self-emulsifying base vehicle, while beneficial for sublingual administration, is versatile and can be utilized for other areas due to its self-emulsifying liposome feature. Thus, beyond administration to the oral cavity, the topical composition it adaptable for administration in nasal, oral, vaginal, topical, and even hair care formulations. Given its multifunctional design, it offers a broad spectrum of possibilities in drug delivery and care solutions, even those beyond active agents, such as one or more GLP-1 receptor agonists, PDE5 inhibitors, selective serotonin receptor agonists, naltrexone, or combination thereof. Indeed, those having skill in the art will appreciate upon reading the present description that embodiments described herein including self-emulsifying base vehicles or that are otherwise formulated to self-emulsify in an aqueous environment may include one or more active agents in addition to or instead of one or more GLP-1 receptor agonists, PDE5 inhibitors, selective serotonin receptor agonists, naltrexone, or combination thereof. Example non-limiting active agents may be selected from analgesics, androgenic hormones, antacids, antianxiety drugs, antiarrhythmics, antibacterials, antibiotics, anticoagulants, antidepressants, antidiarrheals, antiemetics, antifungals, antihistamines, antihypertensives, anti-inflammatoir antineoplastics, antipsychotics, antipyretics, antivirals, anxiolytics, barbiturates, beta-blockers, bronchodilators, cold medicines, corticosteroids, cough suppressants, cytotoxics, decongestants, diuretics, expectorant, hormones, hypoglycemics, immunosuppressives, laxatives, mucolytics, muscle relaxants, sedatives, sleeping aids, thrombolytics, tranquilizer, or vitamins. Such topical compositions may be administered to treat conditions such active agents are used to treat in the art. Such topical compositions may be topically administered as described herein for transdermal or transmucosal delivery. For example, the topical compositions may be administered to the oral cavity, anus, rectum, nasal cavity, ear, or vagina. The topical composition may be administered to external skin surfaces.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular preferred arrangements disclosed for carrying out this invention, but that the invention will include all embodiments and arrangements falling within the scope of the appended claims.

Various elements described herein have been described as alternatives or alternative combinations, e.g., in a list of selectable active agents, ingredients, or compositions. It is to be appreciated that embodiments may include one, more, or all of any such elements. Thus, this description includes embodiments of all such elements independently and embodiments including such elements in all combinations. Any component or ingredient disclosed herein may be explicitly excluded in an embodiment. In various embodiments, the topical composition may be free of biologics, nucleic acids, corn, soy, monosaccharides, disaccharides, milk, collagen, nuts, fish, shellfish, penicillin, hydrocarbons, alcohol, NSAIDs, anticonvulsants, gluten, or combination thereof. In one example, the topical composition is free of sulfonamides, which may be in addition to being free of one or more of the above.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a range is stated as 1 to 50, it is intended that values such as 2 to 40, 10 to 30, 1 to 3, or 2, 25, 39 and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “approximately” or “about” are intended to include +/−10% of the number modified.

What is claimed is:

1. A topical composition for administration to an oral cavity of a subject and oral absorption therein to treat opioid use disorder (OUD) or alcohol use disorder (AUD), the topical composition comprising:
an anhydrous suspension comprising:
naltrexone,
medium chain triglycerides,
between 0.5% w/w and 10% w/w phosphatidylcholine,
between 0.1% w/w and 1% w/w lysophosphatidylcholine,
glyceryl distearate, and
glyceryl monostearate,
wherein the anhydrous suspension self-emulsifies in an aqueous environment of the oral cavity of the subject to form a mixed micelle and liposome delivery system for transmucosal delivery of the sumatriptan within the oral cavity.

2. The topical composition of claim 1, wherein the medium chain triglycerides comprise caprylic/capric triglycerides.

3. The topical composition of claim 2, wherein the caprylic/capric triglycerides are present in an amount between 60% and 95% w/w.

4. The topical composition of claim 2, wherein the caprylic/capric triglycerides are present in an amount between 50% and 81% w/w.

5. The topical composition of claim 1, wherein the anhydrous suspension further comprises polycarbophil in an amount between 0.2% and 2% w/w.

6. The topical composition of claim 1, wherein the anhydrous suspension further comprises polycarbophil in an amount between 0.17% and 2% w/w.

7. The topical composition of claim 1, wherein the anhydrous suspension further comprising peppermint flavor, spearmint flavor, or both.

8. The topical composition of claim 1, wherein the glyceryl distearate and glyceryl monostearate are each present in an amount between 0.5% and 5% w/w.

9. The topical composition of claim 1, wherein the medium chain triglycerides comprise caprylic/capric triglycerides in an amount between 60% and 90% w/w, and wherein the glyceryl distearate and glyceryl monostearate are each present in an amount between 0.5% and 5% w/w.

10. The topical composition of claim 9, wherein the naltrexone is present in an amount between about 3 mg/mL and about 15 mg/mL.

11. The topical composition of claim 10, wherein the phosphatidylcholine is present in an amount between about 1.5% and about 4% w/w and the lysophosphatidylcholine is present in an amount between about 0.28% and about 0.45% w/w.

12. The topical composition of claim 10, wherein the anhydrous suspension further comprises magnesium stearate and microcrystalline cellulose.

13. The topical composition of claim 12, wherein the anhydrous suspension further comprises colloidal silicon dioxide and croscarmellose sodium.

14. A method of treating opioid use disorder (OUD) or alcohol use disorder (AUD) comprising:
administering an anhydrous suspension to an oral cavity for oral absorption therein, the anhydrous suspension comprising:
naltrexone,
medium chain triglycerides,
between 0.5% w/w and 10% w/w phosphatidylcholine,
between 0.1% w/w and 1% w/w lysophosphatidylcholine,
glyceryl distearate, and
glyceryl monostearate,
wherein the anhydrous suspension self-emulsifies in an aqueous environment of the oral cavity of the subject to form a mixed micelle and liposome delivery system for transmucosal delivery of the sumatriptan within the oral cavity.

15. The method of claim 14, wherein the medium chain triglycerides comprise caprylic/capric triglycerides.

16. The method of claim 15, wherein the caprylic/capric triglycerides are present in an amount between 60% and 95% w/w.

17. The method of claim 15, wherein the caprylic/capric triglycerides are present in an amount between 50% and 81% w/w.

18. The method of claim 14, wherein the anhydrous suspension further comprises polycarbophil in an amount between 0.2% and 2% w/w.

19. The method of claim 14, wherein the anhydrous suspension further comprises polycarbophil in an amount between 0.17% and 2% w/w.

20. The method of claim 14, wherein the anhydrous suspension further comprising peppermint flavor, spearmint flavor, or both.

21. The method of claim 14, wherein the glyceryl distearate and glyceryl monostearate are each present in an amount between 0.5% and 5% w/w.

22. The method of claim 14, wherein the medium chain triglycerides comprise caprylic/capric triglycerides in an amount between 60% and 90% w/w, and wherein the glyceryl distearate and glyceryl monostearate are each present in an amount between 0.5% and 5% w/w.

23. The method of claim 22, wherein the naltrexone is present in an amount between about 3 mg/mL and about 15 mg/mL.

24. The method of claim 23, wherein the phosphatidylcholine is present in an amount between about 1.5% and about 4% w/w and the lysophosphatidylcholine is present in an amount between about 0.28% and about 0.45% w/w.

25. The method of claim 23, wherein the anhydrous suspension further comprises magnesium stearate and microcrystalline cellulose.

26. The method of claim 25, wherein the anhydrous suspension further comprises colloidal silicon dioxide and croscarmellose sodium.
